SEQUENCE LISTING (15 sequences)

SEQ ID NO: 1 - DNA, 821 bp, Homo sapiens
  source 1..821: mol_type = other DNA
tcgcgcgcctacactaggcgctgaaatgggatgctggggcttggtggctccggcgggagcagctggtagggctagggctccctggccccccttgaaggggttgggctgcgtgggtgggggctgtgcggggctccgggggccacactcacgccctgtgtcgcccgcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccg

SEQ ID NO: 2 - DNA, 214 bp, Homo sapiens
  source 1..214: mol_type = other DNA
ccgaacaaagaggacaagcggccgcgcacggcctttaccgccgagcagctgcagaggctcaaggccgagttccagaccaacaggtacctgacggagcagcggcgccagagcctggcgcaggagctgagcctcaacgagtcacagatcaagatttggttccagaacaagcgcgccaagatcaagaaggccacgggcaacaagaacacgctggccg

SEQ ID NO: 3 - DNA, 379 bp, Homo sapiens
  source 1..379: mol_type = other DNA
ccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacg

SEQ ID NO: 4 - DNA, 22 bp, synthetic construct
  source 1..22: mol_type = other DNA
  misc_feature 1..22: note = PDX1 Methylation F primer
gattgcgtcgtgattttcggcg

SEQ ID NO: 5 - DNA, 22 bp, synthetic construct
  source 1..22: mol_type = other DNA
  misc_feature 1..22: note = PDX1 Methylation R primer
caaacgacaaacgaccctctcg

SEQ ID NO: 6 - DNA, 22 bp, synthetic construct
  source 1..22: mol_type = other DNA
  misc_feature 1..22: note = EN2 Methylation F primer
ggtatttgacggagtagcggcg

SEQ ID NO: 7 - DNA, 26 bp, synthetic construct
  source 1..26: mol_type = other DNA
  misc_feature 1..26: note = EN2 Methylation R primer
cccgtaaccttcttaatcttaacgcg

SEQ ID NO: 8 - DNA, 27 bp, synthetic construct
  source 1..27: mol_type = other DNA
  misc_feature 1..27: note = MSX1 Methylation F primer
gaagtagtatttgtttatcgtcgagcg

SEQ ID NO: 9 - DNA, 29 bp, synthetic construct
  source 1..29: mol_type = other DNA
  misc_feature 1..29: note = MSX1 Methylation R primer
tcttataatctctttaccttaacgcgacg

SEQ ID NO: 10 - DNA, 28 bp, synthetic construct
  source 1..28: mol_type = other DNA
  misc_feature 1..28: note = PDX1 Unmethylation F primer
gagtaggattgtgttgtgatttttggtg

SEQ ID NO: 11 - DNA, 28 bp, synthetic construct
  source 1..28: mol_type = other DNA
  misc_feature 1..28: note = PDX1 Unmethylation R primer
taaaaccaaacaacaaacaaccctctca

SEQ ID NO: 12 - DNA, 30 bp, synthetic construct
  source 1..30: mol_type = other DNA
  misc_feature 1..30: note = EN2 Unmethylation F primer
gattaataggtatttgatggagtagtggtg

SEQ ID NO: 13 - DNA, 33 bp, synthetic construct
  source 1..33: mol_type = other DNA
  misc_feature 1..33: note = EN2 Unmethylation R primer
cttattacccataaccttcttaatcttaacaca

SEQ ID NO: 14 - DNA, 31 bp, synthetic construct
  source 1..31: mol_type = other DNA
  misc_feature 1..31: note = MSX1 Unmethylation F primer
gttagaagtagtatttgtttattgttgagtg

SEQ ID NO: 15 - DNA, 31 bp, synthetic construct
  misc_feature 1..31: note = MSX1 Unmethylation R primer
  source 1..31: mol_type = other DNA
cctcttataatctctttaccttaacacaaca

(19) United States
(12) Patent Application Publication
(10) Pub. No.: US 2023/0015571 A1
(43) Pub. Date: Jan. 19, 2023

(54) METHOD FOR DIAGNOSING COLORECTAL CANCER BY DETECTING INTRAGENIC METHYLATION

(71) Applicant: Industry-Academic Cooperation Foundation, Yonsei University, Seoul (KR)

(72) Inventors: Lark Kyun Kim, Seoul (KR); Yeongun Lee, Seoul (KR); Jiyeon Lee, Seoul (KR); So Hee Dho, Seoul (KR)

(21) Appl. No.: 17/810,823

(22) Filed: Jul. 5, 2022

(30) Foreign Application Priority Data
Jul. 5, 2021 (KR) 10-2021-0087912
Jul. 5, 2021 (KR) 10-2021-0087933

(51) Int. Cl. C12Q 1/6886

(52) U.S. Cl. CPC C12Q 1/6886; C12Q 1/6858

(57) ABSTRACT

The present invention relates to a method of diagnosing or predicting the prognosis of colorectal cancer by measuring the methylation level in the intragenic region of PDXJ, EN2 and/or MSXJ. The present invention provides highly reliable biomarkers for colorectal cancer by identifying CpG regions in genes that are hypermethylated specifically in colorectal cancer patients, and also provides optimized methylation-specific PCR (MSP) primers capable of efficiently detecting the identified CpG regions. Accordingly, the present invention may provide important clinical information that makes it possible to accurately predict not only the onset of colorectal cancer, but also overall prognosis including the degree of invasion of cancer tissue, the likelihood of metastasis, and the survival rate of the patient, thereby establishing a treatment strategy early and significantly improving the survival rate of colorectal cancer patients. The present invention also provides, as guidelines for the design of primers capable of accurately detecting DNA methylation, optimal parameters for the amplicon length, the total number of CpGs in target gene-binding regions of the primers, and the range of Tm values.

### CROSS-REFERENCE TO RELATED APPLICATIONS

This application claims the benefit of priority of Korean Patent Application no. 10-2021-0087912, filed Jul. 5, 2021, and Korean Patent Application no. 10-2021-0087933, filed Jul. 5, 2021, each of which is hereby incorporated herein by reference in its entirety.

### SEQUENCE LISTING

A computer readable form of the Sequence Listing is submitted via a USPTO patent electronic filing system with this application and is incorporated into this application by reference in its entirety. The Sequence Listing is contained in the sequence listing XML file created on Jul. 5, 2022, having the file name “214 Sequence-Listing.xml” and is 20 kb in size.

### BACKGROUND

### 1. Technical Field

In various aspects, the present invention relates to a method of predicting the onset or prognosis of colorectal cancer by measuring the methylation level in the intragenic region of PDX1 gene, EN2 gene and/or MSX1 gene. In various aspects, the present invention also relates to a nucleic acid molecule for detecting a DNA methylation variation region in a target gene, specifically a methylation-specific PCR (MSP) primer set, and a method for producing the same.

### 2. Related Art

Colorectal cancer (CRC) is the third most common cancer worldwide, accounting for the second-highest mortality in 2020^{[1]}. CRC is widely known to occur due to the accumulation of genetic and epigenetic alterations. Several molecular pathways involved in the onset and development of CRC have been identified, including the adenoma—carcinoma pathway (also called the chromosomal instability sequence), the serrated neoplasia pathway, and microsatellite instability (MSD^{[2,3]}. The adenoma—carcinoma pathway accounts for 70-90% of CRC cases and is generally initiated by APC mutations, followed by KRAS activation or loss of TP53 function. Conversely, the serrated neoplasia pathway develops via KRAS and BRAF mutations, and epigenetic dysregulation is uniquely distinguished by the CpG island methylator phenotype (CIMP). MSI typically occurs with Lynch syndrome, mainly due to mismatch repair (MMR) gene inactivation^{[4-7]}.

Among the epigenetic modifications in mammals, DNA methylation plays a key role in regulating gene expression. This epigenetic regulation affects tumor suppressor gene and oncogene expression, and this mode of action is slightly different among cancer types. DNA methylation markers have been extensively studied in CRC. Because of the hypomethylation and activation of repetitive sequences, such as long interspersed nuclear element-1 (LINE-1) and Alu repeats, genomic instability is thought to occur and could boost CRC initiation ^{[11-13]}Conversely, a panel of genomic regions and genes hypermethylated in promoter regions was found and was later identified as a type of CRC called CIMP^{[14]}. In general, gene expression is decreased when DNA hypermethylation occurs in the promoter of a gene, and thus hypermethylated genes of the CIMP are expected to function as tumor suppressors.

Despite numerous observations regarding the relationship between DNA methylation changes and cancer progression, only a few genes, such as SEPT9 (Epi proColon), NDRG4, and BMP 3 (Cologuard), have been verified as diagnostic CRC biomarkers and have been approved for commercialization via diagnostic kits^{[15-17]}. The cornerstone of developing DNA methylation-based biomarkers is the selection of ideal genomic locations, that is, CpG islands (CGIs) and specific CpG sites^{[19]}. For example, DNA methylation in the promoter region of GSTP1 has been identified as a promising diagnostic marker for hepatocellular carcinoma but with conflicting variation in terms of its specificity.

It was later discovered that this variability resulted from differences in the CpG sites of the 5′ region of the GSTP1 promoter used for measuring DNA methylation levels^{[20]}. In other words, this suggests that detection sensitivity and clinical relevance may vary depending on how the CpG sites within the same CpG island are selected.

To discover clinical biomarkers based on next-generation sequencing technology, Illumina Infinium 450 K or 850 K array-based detection methods have been used for massive data generation by The Cancer Genome Atlas (TCGA)^{[21]}. This method makes it possible to screen and observe the methylation levels of various genes in cancer cells. Whole-genome bisulfite sequencing is a powerful method capable of determining DNA methylation levels on a genome-wide scale but has significant limitations in terms of time and cost. Targeted sequencing technology may be used for the high-throughput sequencing of genomic regions of interest. To increase the specificity of the quantification of DNA methylation, targeted bisulfite sequencing utilizes probes designed to bind and capture target regions for PCR-based enrichment.

However, a more straightforward methylation method, methylation-specific polymerase chain reaction (MS-PCR, MSP), has been developed^{[23]}, and this method may measure methylation in target regions in a time- and cost-effective way, but it is relatively difficult to design primers therefor and optimize PCR conditions^{[24]}.

Throughout the specification, a number of publications and patent documents are referred to and cited. The disclosure of the cited publications and patent documents is incorporated herein by reference in its entirety to more clearly describe the state of the related art and the present invention.

### PRIOR ART DOCUMENTS

### Non-Patent Documents

Non-Patent Document 1. Kel et al. Walking pathways with positive feedback loops reveal DNA methylation biomarkers of colorectal cancer BMC Bioinformatics 20:119 (2019).

### SUMMARY

The present inventors have made intensive research efforts to develop a method of accurately predicting the onset and prognosis of colorectal cancer based on epigenetic genetic changes in individuals. As a result, the present inventors have found that, when the DNA methylation pattern in the intragenic region of PDX1 gene, EN2 gene and/or MSX1 gene is measured, it is possible to predict not only the current onset of colorectal cancer, but also overall prognosis including the degree of invasion of cancer tissue, the possibility of metastasis, and the survival rate of the patient, with high reliability, thereby completing various aspects of the present invention.

Therefore, one object of various aspects of the present invention is to provide a composition for diagnosing or predicting the prognosis of colorectal cancer.

Another object of various aspects of the present invention is to provide a nucleic acid molecule for detecting methylation of a target gene.

Other objects and advantages of various aspects of the present invention will be more apparent by the following detailed description of the invention, the claims and the accompanying drawings.

According to one aspect of the present invention, the present invention provides a composition for diagnosing or predicting the prognosis of colorectal cancer, the composition containing, as an active ingredient, an agent for measuring the methylation level in the intragenic region of at least one gene selected from the group consisting of PDX1, EN2 and

MSX1 genes.

The present inventors have made intensive research efforts to develop a method of accurately predicting the onset and prognosis of colorectal cancer based on epigenetic genetic changes in individuals. As a result, the present inventors have found that, when the DNA methylation pattern in the intragenic region of PDX1 gene, EN2 gene and/or MSX1 gene is measured, it is possible to predict not only the current onset of colorectal cancer, but also overall prognosis including the degree of invasion of cancer tissue, the possibility of metastasis, and the survival rate of the patient, with high reliability.

As used herein, the term “colorectal cancer” refers to a malignant tumor occurring in the large intestine, which is a digestive organ located between the small intestine and anus. Colorectal cancer occurs mainly in the epithelial cells of the large intestine, and is classified into colon cancer and rectal cancer depending on the location of the cancer. Thus, the term “colorectal cancer” is meant to encompass “colon cancer”, “rectal cancer” and “colorectal cancer (CRC)”.

As used herein, the term “diagnosis” is meant to encompass determination of subject's susceptibility to a specific disease, determination of whether a subject currently has a specific disease, and determination of the prognosis of a subject having a specific disease and the therapeutic responsiveness of the subject to a specific drug.

In the present specification, the term “composition for diagnosing” refers to an integrated mixture or device comprising a means for measuring the methylation level of PDX1, EN2 and/or MSX1 gene in order to determine whether or not a subject has developed colorectal cancer or to predict the likelihood of developing colorectal cancer. Thus, this term may also be referred to as a “diagnostic kit”.

As used herein, the term “prognosis” is meant to encompass the prediction of disease symptoms or progression after analysis, determined by diagnosing the disease. Prognosis in colorectal cancer patients usually refers to whether or not metastasis occurs within a certain period of time after cancer onset or surgical procedure, and the overall survival time and the disease-free survival rate of the patients. Prediction of prognosis provides clues to the future treatment strategy for colorectal cancer patients, and thus is an important clinical task.

As used herein, the term “disease-free survival rate” or “progression-free survival rate” refers to the proportion of patients, who survived without an increase in recurrence or metastasis for 5 years after starting treatment, in the entire patient group. The term “metastasis” refers to a condition in which a tumor spreads from a primary site to other part of the body along multiple routes and engrafts and proliferates therein. Since whether cancer has metastasized is not only determined by the intrinsic characteristics of the cancer, but also is an event that is the most important clue in determining the prognosis of the cancer, it is regarded as the most important clinical information associated with the survival of cancer patients.

As used herein, the term “therapeutic responsiveness” refers to the degree to which a therapeutic agent, administered to a patient in a therapeutically effective amount, acts to suppress the progression of, alleviate or eliminate disease symptoms in vivo.

According to various aspects of the present invention, the present inventors have found first that the methylation in the intragenic region of PDX1, EN2 and/or MSX1 is significantly higher in tumor tissue than in normal tissue, and this hypermethylation may serve as a marker for the onset of colorectal cancer, increased proliferation, migration and invasion of tumor cells, and patient survival rate.

According to a specific embodiment of the present invention, the agent for measuring the methylation level in the intragenic region of PDX1 is a methylation-specific PCR (MSP) primer set that specifically recognizes the intragenic CpG island of PDX1.

As used herein, the term “primer” refers to an oligonucleotide that acts a point of initiation of synthesis when placed under conditions in which the synthesis of a primer extension product complementary to a nucleic acid strand (template) is induced, i.e., in the presence of nucleotides and an agent for polymerization, such as DNA polymerase, and at a suitable temperature and pH. Specifically, the primer is a single-stranded deoxyribonucleotide. The primers that are used in the present invention may include naturally occurring dNMPs (i.e., dAMP, dGM, dCMP and dTMP), modified nucleotide, or non-natural nucleotide. The primers may also include ribonucleotides.

Various primers of the present invention may be an extension primer that is annealed to the target nucleic acid to form a sequence complementary to the target nucleic acid by a template-dependent nucleic acid polymerase. It extends to a position where an immobilization probe is annealed and occupies the area where a probe is annealed.

The extension primer used in various aspects of the present invention comprises a hybridization nucleotide sequence complementary to a target nucleic acid, for example, a specific nucleotide sequence of an intragenic CpG island in PDXJ, EN2 and/or MSX1 gene. As used herein, the term “complementary” means that the primer or probe is sufficiently complementary to hybridize selectively to the target nucleic acid sequence under certain annealing or hybridization conditions. The term is meant to encompass both substantially complementary and perfectly complementary, and preferably means perfectly complementary. As used herein, the term “substantially complementary sequence” is meant to encompass not only a perfectly matching sequence but also a sequence partially mismatching with the target sequence within a range in which the sequence may be annealed to a specific sequence and serve as a primer.

The primer must be sufficiently long to prime the synthesis of extension products in the presence of the agent for polymerization. The suitable length of each primer will depend on various factors, such as temperature, pH and the source of the primer, but is typically 15 to 30 nucleotides. Short primer molecules generally require cooler temperatures to form sufficiently stable hybrid complexes with template. Short primer molecules generally require cooler temperatures to form sufficiently stable hybrid complexes with template. The design of these primers can be easily performed by those skilled in the art with reference to the target nucleotide sequence, and for example, it may be performed using a primer design program (e.g., PRIMER 3 program).

As used herein, the term “methylation-specific PCR (MSP) primer set” refers to a primer set for use in PCR which is performed to obtain information on the DNA methylation status in a target nucleic acid molecule. MSP may be performed by treating a DNA molecule to be analyzed with sodium bisulfite to convert unmethylated cytosine in the DNA molecule into thymine, and then performing PCR with primers capable of binding specifically to the CpG island of a gene (e.g., PDXJ, EN2 and/or MSX1 ) to be analyzed, which has a modified nucleotide sequence. Thus, the term “MSP primer set” means a primer set designed to distinguish between unmethylated cytosine and methylated cytosine in consideration of whether cytosine is converted to thymine by sodium bisulfite treatment.

In various aspects of the present invention, a probe may be used together with the primer set.

As used herein, the term “probe” refers to linear oligomers having natural or modified monomers or linkages, including deoxyribonucleotides and ribonucleotides, which can hybridize to a specific nucleotide sequence. Specifically, the probe is single-stranded for maximum efficiency in hybridization. More specifically, the probe is deoxyribonucleotide. As the probe used in the present invention, a sequence perfectly complementary to a specific nucleotide sequence of the intragenic CpG island of PDXJ, EN2 and/or MSX1 gene may be used, but a substantially complementary sequence may also be used within a range that does not interfere with specific hybridization. In general, the stability of a duplex formed by hybridization tends to be dependent on the sequence matching of terminal sites, and thus it is preferable to use a probe complementary to the 3′-end or 5′-end of the target sequence.

Suitable conditions for hybridization may be determined with reference to Joseph Sambrook et al., Molecular Cloning, A Laboratory Manual, Cold Spring Harbor Laboratory Press, N.Y. (2001), and Haymes, B. D., et al., Nucleic Acid Hybridization, A Practical Approach, IRL Press, Washington, D.C. (1985).

In various more specific embodiments, the intragenic CpG island of PDX1 comprises the nucleotide sequence of SEQ ID NO: 1 (chr13: 28,498,226-28,499,046) in the sequence listing filed herewith, which sequence listing is hereby incorporated herein by reference in its entirety.

In various more specific embodiments, the primer for MSP is a pair of primers comprising the nucleotide sequence of SEQ ID NO: 4 and the nucleotide sequence of SEQ ID NO: 5, respectively.

According to a specific embodiment of the present invention, the agent for measuring the level of methylation in the intragenic region of EN2 is a methylation-specific MSP primer set that specifically recognizes the intragenic CpG island of EN2.

In various more specific embodiments, the intragenic CpG island of EN2 comprises the nucleotide sequence of SEQ ID NO: 2 (chr7: 155,255,098-155,255,311).

More specifically, the MSP primer set is a pair of primers comprising the nucleotide sequence of SEQ ID NO: 6 and the nucleotide sequence of SEQ ID NO: 7, respectively.

According to a specific embodiment of the present invention, the agent for measuring the level of methylation in the intragenic region of MSX1 is a methylation-specific MSP primer set that specifically recognizes the intragenic CpG island of MSX1 .

More specifically, the intragenic CpG island of MSX1 comprises the nucleotide sequence of SEQ ID NO: 3 (chr4: 4,864,456-4,864,834).

In various more specific embodiments, the MSP primer set is a pair of primers comprising the nucleotide sequence of SEQ ID NO: 8 and the nucleotide sequence of SEQ ID NO: 9, respectively.

As used herein, the term “nucleotide” is meant to encompass DNA (gDNA and cDNA) and RNA molecules, and nucleotides, which are basic structural units in the nucleic acid molecule, include not only natural nucleotides but also analogues with modified sugar or base moieties. It is obvious to those skilled in the art that the nucleotide sequences of the regions whose methylation level is to be measured in the present invention or the primer sequences for MSP to be used for measurement of the methylation level are not limited to the nucleotide sequences described in the accompanying sequence list. Considering nucleotide variants having biologically equivalent activities, it is interpreted that the intragenic CpG island of each marker gene and the MSP primer set of the present invention also include a sequence showing substantial identity to the sequence described in the sequence list. The term “sequences substantially identical” refers to sequences showing at least 70%, specifically at least 80%, more specifically at least 90%, most specifically at least 95% similarity to the sequence of the present invention, when aligning any other sequences with the sequence of the present invention so as to correspond to each other to the highest possible extent and analyzing the aligned sequences using algorithms that are generally used in the art. Methods of alignment of sequences for comparison are well-known in the art. Alignment methods for sequence comparison are known in the art. Various methods and algorithms for alignment are described in Huang et al., Comp. Appl. BioSci. 8:155-65 (1992) and Pearson et al., Meth. Mol. Biol. 24:307-31 (1994). The NCBI Basic Local Alignment Search Tool (BLAST) (Altschul et al., J. Mol. Biol. 215:403-10 (1990)) is available from the NCBI (National Center for Biological Information) or the like and may be used in conjunction with sequencing programs, such as blastp, blasm, BLASTX, tblastn and tblastx, on the Internet.

As used herein, the term “subject” refers to a subject that provides a sample for measuring the intragenic methylation level of each marker gene, and ultimately refers to a subject to be analyzed for diagnosis and prognosis of colorectal cancer. Examples of the subject includes, without limitation, humans, mice, rats, guinea pigs, dogs, cats, horses, cattle, pigs, monkeys, chimpanzees, baboons or rhesus monkeys, specifically humans. Since the composition of the present invention provides information for predicting not only whether colorectal cancer has developed but also the genetic risk of future development, metastasis and recurrence of colorectal cancer, the subject of the present invention may be a colorectal cancer patient or may also be a healthy subject that has not yet developed colorectal cancer.

According to another aspect of the present invention, the present invention provides a composition for diagnosing colorectal cancer containing, as an active ingredient, an agent for measuring the expression level of at least one gene selected from the group consisting of PDX1, GRIN2D, PITX1, TFAP2A, EN2 and MSX1 genes.

The present inventors identified CpG islands in intragenic regions showing methylation patterns different between normal samples and tumor samples, and then further investigated the effect of these methylation patterns on the expression levels of the corresponding genes. As a result, the present inventors have found that the expression levels of the six genes listed above increase two-fold or more in colorectal cancer tissue, suggesting that these genes can function as effective diagnostic markers for colorectal cancer.

According to still another aspect of the present invention, the present invention provides a composition for predicting the prognosis of colorectal cancer, the composition containing, as an active ingredient, an agent for measuring the expression level of at least one gene selected from the group consisting of PDXJ, EN2 and MSX1 genes.

As shown in the Examples described below, the present inventors have found that high expression of PDXJ, EN2 and MSX1 genes promotes the proliferation, invasion and migration of colorectal cancer cells and is negatively correlated with patient survival, suggesting that these genes can function as highly reliable biomarkers for predicting the prognosis of colorectal cancer.

According to a specific embodiment of the present invention, the composition is a composition for predicting metastasis of colorectal cancer.

As used herein, the term “metastasis” or “metastatic cancer” refers to a new cancer formed as cancer cells detached from the primary tumor tissue penetrate into surrounding blood vessels or lymphatic vessels and move to other remote parts of the body through the blood vessels or lymphatic vessels. Since 90% or more of cancer patient deaths are due to metastasis from primary cancer (Nature Reviews Cancer, 2006, 6:449-458), early detection of cancer metastasis is as important as the treatment of primary cancer in improving the survival rate of cancer patients.

The present inventors have found that not only the methylation levels of the intragenic CpG regions in PDXJ, EN2 and MSX1 genes but also the expression levels of these genes have a significant relationship with cancer cell migration, and thus the expression levels of these genes can be markers for cancer cell metastasis to secondary sites. Accordingly, “prediction of metastasis” is used in the same sense as “diagnosis of metastatic cancer”, “diagnosis of cancer metastasis” or “prediction of prognosis of cancer”.

According to yet another aspect of the present invention, the present invention provides a nucleic acid molecule for detecting methylation in a target gene, the nucleic acid molecule comprising forward and reverse primers which form an amplicon having a length of 90 bp to 170 bp, comprise a total of 6 to 9 CpG sites in target gene-binding regions thereof, and have a melting temperature (Tm) of 53 to 62° C.

The present inventors have made intensive research efforts to develop a method of accurately and efficiently detecting a change in expression of a specific gene or individual's epigenetic genetic changes associated with the progression of specific diseases including cancer. As a result, the present inventors have found that, when a primer set for measuring the DNA methylation level of a target gene is designed by setting the amplicon length to 90 bp to 170 bp, the total number of CpGs in target gene-binding regions thereof to 6 to 9, and the Tm value range to 53 to 62° C., the degree of methylation in the target region can be detected with high reliability and with the highest sensitivity within the corresponding parameter ranges.

As expression of genes is restored or suppressed due to downregulated methylation or hypermethylation of genomic DNA, attempts have been actively made to regulate the expression of oncogenes, metastatic genes, and anticancer drug resistance genes. Accordingly, obtaining accurate information on the methylation level of a target region is emerging as an important task for establishing a patient diagnosis and treatment strategy.

As described above, the forward and reverse primers of various aspects of the present invention are designed to form an amplicons of 90 bp to 170 bp. More specifically, the primers are designed to form an amplicon of 95 bp to 165 bp, most specifically 100 bp to 160 bp.

As described above, the primers of various aspects of the present invention have a melting temperature (Tm) of 53 to 62° C., more specifically 54 to 61° C., most specifically 55 to 60° C.

According to a specific embodiment of the present invention, the forward and reverse primers have a length of 20 bp to 35 bp, more specifically 21 bp to 34 bp, most specifically 22 bp to 33 bp.

According to a specific embodiment of the present invention, the melting temperature (Tm) difference between the forward and reverse primers is less than 2° C.

The primer set of various aspects of the present invention can measure not only the methylation level in the intragenic region of the target gene, but also methylation of an expression control sequence such as a promoter in the target gene. Specifically, the primer set of the present invention measures the methylation level in the intragenic region of the target gene.

According to a specific embodiment of the present invention, the primer set is a methylation-specific PCR (MSP) primer set that specifically recognizes an intragenic CpG island.

As described above, primers useful in various embodiments of the present invention comprise a total of 6 to 9 CpG sites in target gene-binding regions thereof, which means that the sum of CpG sites in the binding regions of the reverse and forward primers is 6 to 9. In more specific embodiments, each primer comprises 6 to 8 CpG sites, for example, 6 or 7 CpG sites.

According to a specific embodiment of the present invention, the methylation level in the intragenic CpG island is different between a patient with a disease and a normal person.

DNA methylation plays a key role in regulating gene expression, thereby affecting the expression of tumor suppressor genes and oncogenes, thereby being involved in suppression of the onset and progression of cancer in an individual.

In more specific embodiments, the disease is cancer, most specifically colorectal cancer.

As noted above, various aspects of the invention provide methods for diagnosing or predicting prognosis of colorectal cancer; methods for diagnosing colorectal cancer; and methods for predicting prognosis of colorectal cancer. And it is noted that these methods can provide important clinical information that makes it possible to accurately predict not only the onset of colorectal cancer, but also overall prognosis including the degree of invasion of cancer tissue, the likelihood of metastasis, and the survival rate of the patient, thereby establishing a treatment strategy early and significantly improving the survival rate of colorectal cancer patients.

Other aspects of the invention relate to methods for treatment of colorectal cancer in a subject. For example, one aspect of the invention is a method for treating colorectal cancer in a subject, the method including: diagnosing or predicting prognosis of colorectal cancer in the subject by a method as described herein; and then based on the diagnosis or prediction of prognosis, treating the subject for colorectal cancer. Another aspect of the invention is a method for treating colorectal cancer in a subject, the method including: diagnosing colorectal cancer in the subject by a method as described herein; and then based on the diagnosis, treating the subject for colorectal cancer. And another aspect of the invention is method for treating colorectal cancer in a subject, the method including: predicting prognosis of colorectal cancer colorectal cancer in the subject by a method as described herein; and then based on the predicted prognosis, treating the subject for colorectal cancer.

There are a variety of treatments for colorectal cancer, and the person of ordinary skill in the art can select an appropriate course of treatment based on the diagnoses and/or predictions of prognoses provided by the methods described herein. In various embodiments, the treatment can include surgery, e.g., to remove colorectal cancer tissue, be it in the colon or rectum, or metastasized tissue remote from the colon or rectum. Surgery may also be used to remove a portion of the colon, and/or one or more lymph nodes. Ablation and/or embolization treatments can also be used. Polyps can be removed surgically or during a colonoscopy, be they cancerous, pre-cancerous or benign. The treatment can, additionally or alternatively, include treatment with one or more colorectal cancer drugs, including, for example, one or more of bevacizumab; irinotecan hydrochloride; capecitabine; cetuximab; ramucirumab; oxaliplatin; 5-fluorouracil; ipilimumab; pembrolizumab; leucovorin calcium; trifluridine and tipiracil hydrochloride; nivolumab; panitumumab; ramucirumab; regorafenib; ipilimumab; Ziv-Aflibercept. Particular combinations include capecitabine and oxaliplatin; leucovorin calcium, 5-fluorouracil and irinotecan hydrochloride; leucovorin calcium, 5-fluorouracil, irinotecan hydrochloride and bevacizumab; leucovorin calcium, 5-fluorouracil, irinotecan hydrochloride and cetuximab; leucovorin calcium, 5-fluorouracil and oxaliplatin; leucovorin calcium and 5-fluorouracil; irinotecan hydrochloride and capecitabine; and capecitabine and oxaliplatin. Radiation treatment can also be used. The person of ordinary skill in the art can select any combination of these treatments, based on the information provided by the methods for diagnosis and/or prediction of prognosis provided by the methods described herein.

Various non-limiting features and advantages of various aspects of the present invention are summarized as follows:

(a) Various aspects of the present invention provide a method of diagnosing or predicting the prognosis of colorectal cancer by measuring the methylation level in the intragenic region of PDXJ, EN2 and/or MSXJ.

(b) Various aspects of the present invention provide highly reliable biomarkers for colorectal cancer by identifying CpG regions in genes that are hypermethylated specifically in colorectal cancer patients, and also provides optimized methylation-specific PCR (MSP) primers capable of efficiently detecting the identified CpG regions.

(c) Various aspects of the present invention may provide important clinical information that makes it possible to accurately predict not only the onset of colorectal cancer, but also overall prognosis including the degree of invasion of cancer tissue, the likelihood of metastasis, and the survival rate of the patient, thereby establishing a treatment strategy early and significantly improving the survival rate of colorectal cancer patients.

(d) Various aspects of the present invention also provide, as guidelines for the design of primers capable of accurately detecting DNA methylation, optimal parameters for the amplicon length, the total number of CpGs in a region binding to a target gene, and the range of Tm values.

(e) Various aspects of the disclosure provide methods for treating colorectal cancer, based on the diagnoses and predictions of prognoses described herein.

### BRIEF DESCRIPTION OF THE DRAWINGS

FIG. 1 is a schematic view showing a process of selecting a cohort-specific DNA methylation biomarker in colorectal cancer. Illumina Infinium 450 K array data of five major gastroenterological cancers (COAD, READ, LIHC, STAD, and PAAD) downloaded from TCGA are preprocessed (Box A). Then, 10,754 differentially methylated CpG islands (CGIs) are shortlisted based on criteria (Box B), and then the hybridizing probe pool targeting selected CGIs is designed using NimbleDesign (Box C). Targeted bisulfate sequencing is conducted for 104 CRC patients from the South Korean cohort (Box D). Generated targeted bisulfite sequencing data are analyzed to select differentially methylated regions (DMRs) in tumors relative to healthy tissues (Box E).

FIGS. 2A, 2B, 2C and 2D show the results of specifying candidate DNA methylation biomarker genes based on differential gene expression and correlation with CRC patient survival outcomes. As shown in FIG. 2A, genomic location analysis of differentially methylated CGIs in targeted bisulfite sequencing data indicates that most hypermethylated regions are evenly distributed between the promoter and intragenic regions, while a larger proportion of hypomethylated regions are in intragenic regions. The present inventors focused on hypermethylated intragenic regions. In FIG. 2B, the expression data (read counts) downloaded from TCGA were examined to identify upregulated genes in tumor samples. Downloaded RNAseq data were processed with DESeq2. FIG. 2C shows gene expression representation of seven upregulated candidate genes in terms of TPM. Their differential expression status was further verified, and genes with nonsignificant differences were omitted from downstream analysis. ns: nonsignificant, *p<0.05, **p<0.01, ***p<0.001. In FIG. 2D, Kaplan—Meier survival plots of the six upregulated genes indicated the difference between patients with high expression of the shortlisted genes (top 25%) and patients with low or medium expression (bottom 75%). Gene expression and clinical data were based on TCGA-COAD.

FIGS. 3A and 3B show that selected candidate DNA methylation biomarker genes drive oncogenic properties by promoting cell proliferation and cell migration in vitro. In FIG. 3A, the cell proliferation test with CCK-8 reagent indicated that overexpression of PDX1, EN2, and MSX1 promotes proliferation of the HCT116 colorectal cancer cell line. The overexpression of each gene was verified through FLAG-tag capture. FIG. 3B shows the results of Transwell invasion assays conducted with HCT116 cells overexpressing PDX1,

EN2 and MSX1. Overexpression of PDX1, EN2 and MSX1 was found to accelerate migration and confer invasive properties.

FIGS. 4A, 4B, 4C, 4D, 4E, 4F, 4G, 4H, and 4I show optimized benchmark for primer-binding site selection and primer design in methylation-specific PCR (MSP). FIGS. 4A, 4B and 4C show MSP-targeting genomic regions in the intragenic CpG islands of PDX1 (FIG. 4A), EN2 (FIG. 4B) and MSX1 (FIG. 4C) (yellow BOX). Hierarchical clustering of healthy tissue and tumor samples of targeted bisulfite sequencing data confirmed the hypermethylation of each target region in the tumor tissue relative to healthy tissues. Each column corresponds to the cytosine of CpG sites within the respective intragenic CpG islands of PDX1, EN2 and MSX1 . Low-quality sequencing data were then filtered out. FIGS. 4D, 4E and 4F show the results of validating the efficacy of methylation detection and quantification of manually designed MSP primers in vitro, in which three colon cancer cell lines (SW480, LoVo, HCT116) and one healthy colon cell line (CCD-18Co) were used. Agarose gel electrophoresis of quantitative MSP (qMSP) products also confirmed the methylation level detection efficacy of the designed primers. FIGS. 4G, 4H and 41 show the results of performing qMSP with CCD-18Co and SW480 template DNA to verify DNA quantity-dependent signal changes of PDX1 (FIG. 4G), EN2 (FIG. 4H), and MSX1 (FIG. 41) methylation. Met: MSP primer that binds to genomic DNA where all the target CpG sites are methylated. Half-Met: the MSP primer that binds with genomic DNA where some of the target CpG sites are methylated. Unmet: MSP primer that binds with genomic DNA where all the target CpG sites are not methylated. nd: not determined. *p<0.05, **p<0.01, ***p<0.001.

FIGS. 5A, 5B, 5C, 5D, 5E, 5F and 5G show that customized MSP primers detect methylation changes in SW480 candidate biomarkers modulated by the CRISPR/dCas9-gRNA system. FIG. 5A is a representation of a CRISPR/dCas9-gRNA system used in the present invention to induce demethylation. FIGS. 5B, 5D and 5F show the results of qMSP with SW480 cells transfected with dCas9-TET1CD mock or gRNA specific to PDX1 (FIG. 5B), EN2 (FIG. 5D) and MSX1 (FIG. 5F), and indicates that the designed primers can distinguish the lack of methylation modulated by the CRISPR/dCas9-gRNA system. FIGS. 5C, 5D and 5G show the results of qPCR with SW480 cells transfected with dCas9-TET1CD mock or gRNA of PDX1 (FIG. 5C), EN2 (FIG. 5E) and MSX1 (FIG. 5G), and indicate that decreased methylation leads to a reduction in gene expression. Genomic DNA and RNA used in qMSP and qPCR were simultaneously extracted from the cell lines.

FIGS. 6A, 6B and 6C show the results of analyzing the potential of the 3-gene methylation signature as a prognostic marker. FIG. 6A shows the results of a Hierarchical clustering conducted with DNA methylation data of intragenic CpG islands of PDXJ, EN2, and MSX1 , where two distinct subgroups of CRC patients were observed. FIGS. 6B and 6C are Kaplan—Meier plots for analyzing the significant differences in overall survival (FIG. 6B) and CRC recurrence (FIG. 6C) between the subgroups, which reveal that the methylation data of the three biomarkers are effective as a prognostic marker. The log-rank test was used to compare the significant differences between the two subgroups. One sample was excluded from the analysis of clinical data due to missing clinical data. Additionally, 31 patients were excluded from the recurrence analysis because they were diagnosed with stage IV CRC with metastatic cancers. FIG. 6D shows qMSP data generated with genomic DNA originating from the tumor and healthy tissues of the seven CRC patients. The qMSP data displayed similar patterns to the cohort-specific methylation change analysis in FIG. 6A. The relative methylation levels of intragenic CpG islands of PDXJ, EN2 and MSX1 were calculated by dividing the methylation level of the tumor by that of healthy tissue.

FIG. 7 shows TCGA Illumina 450K array data preprocessing. The Illumina Infinium 450 K microarray data from The Cancer Genome Atlas were downloaded from the GDC data portal of the National Institutes of Health (NIH). Each sample contained a beta value of approximately 450,000 probes. To estimate the methylation value of CpG islands, CpG sites on the same CpG island according to hg19 were averaged. Differences in methylation values between tumors and an average of healthy tissues were calculated, and CpG islands in which methylation levels differed by >20% between tumors and the average of healthy tissues in 20% or more of the total patients were selected. A total of 10,754 CpG islands showed differential methylation, and were used to design a targeting probe pool.

FIG. 8 shows a process of preparing a targeted DNA methylation sequencing library. Genomic DNA from healthy and tumor tissues from the CRC cohort was extracted. Only QC-passed samples were used for the preparation of the targeted bisulfite sequencing library. Each genomic DNA was sheared to 250 to 300 bp, which is the gold standard for high-throughput sequencing. Single-stranded ends of sheared genomic DNA were repaired, followed by A-tailing, adaptor ligation, and size selection. Bisulfite conversion of genomic DNA was conducted to differentiate unmethylated cytosines from their methylated counterparts. To recover an appropriate quantity of bisulfite-converted genomic DNA, PCR amplification was performed after hybridization. After each amplification step, the quality and quantity of the PCR products were confirmed using the Agilent 2100 Bioanalyzer system. The prepared samples were then used for high-throughput sequencing using Hiseq2500.

FIG. 9 is a schematic view showing targeted bisulfite sequencing data preprocessing. Trimgalore (ver. 0.5.0) was used to trim the adaptor sequence from each targeted bisulfite sequencing data, and sequencing reads were aligned on the hg19 human genome reference using Bismark and Bowtie2. The sequencing reads were then sorted and indexed, and their methylation counts were extracted. CpG sites with a read depth below 10 were filtered out. Methylation values of CpG sites were averaged to estimate the methylation values of CpG islands.

FIG. 10 is a schematic view showing TCGA RNA-seq data pre-processing. Count data aligned by HT-seq were downloaded. Each RNA-seq data was integrated into a matrix, and gene expression differences between tumor and healthy tissues were calculated using DESeq2. To obtain normalized gene expression data (TPM value), the scaled-estimate value of RNA-seq data aligned by STAR was multiplied by 10⁶.

FIGS. 11A and 11B show the expression levels of candidate methylation biomarker genes. The TPM values of the genes from Table 1 are listed. RNA-seq data of candidate genes from healthy and tumor colon tissues were downloaded from TCGA, and TPM values were calculated by multiplying the scaled-estimate value of RNA-seq data by 10⁶.

FIGS. 12A, 12B and 12C shows MSP targeting genomic regions in the intragenic CpG islands of PDXJ, EN2, and MSXJ. FIGS. 12A, 12B and 12C each show a line graph indicating the average DNA methylation level of the CpG sites in the candidate CpG island and their targeted MSP primer binding sites. Targeted bisulfite sequencing data were used in the plotting process. In the left panel of each figure, the red line represents the average methylation level of healthy samples, while the blue line corresponds to tumor samples. Each dot in the line graph denotes the CpG sites included in the CpG island. The yellow boxes indicate the MSP forward and reverse primer binding sites. The right panel of each figure shows DNA methylation status of CpG sites in healthy and tumor colon tissues. Each dot represents the CpG site, and the dark portion of each dot represents the average methylation level.

FIGS. 13A, 13B and 13C show the results of subcloning of pPlatTET-gRNA2. FIG. 13A is the design scheme of subcloning gRNA in the dCas9-TET1CD vector. FIG. 13B shows pyrosequencing results for subcloned vectors, and each gRNA coding sequence was validated through manual inspection. FIG. 13C shows GFP expression in SW480 cells transfected with dCas9-TET1CD vectors.

FIG. 14 shows methylation patterns in HOXA3-related CpG islands from generated targeted bisulfite sequencing data. It shows the DNA methylation level of 7 CpG islands in HOXA3 (chr7:27,163,819-27,164,098, chr7:27,162,087-27,162,426, chr7:27,154,999-27,155,426, chr7:27,153,187-27,153,647, chr7:27,150,030-27,150,418, chr7:27,147,589-27,148,389, and chr7:27,146,069-27,146,600). The human genome reference version used was hg19, and data were visualized using the IGV browser. The bar graph indicates the average methylation level of CpG sites in each CpG island.

FIG. 15 shows methylation patterns in BCAT1-related CpG islands from targeted bisulfite sequencing data. It shows DNA methylation levels of two CpG islands in BCAT1 (chr12:25,101,607-25,102,073, and chr12:25,055,599-25,056,246). The human genome reference version used was hg19, and data were visualized using the IGV browser. The bar graph indicates the average methylation level of CpG sites in each CpG island.

FIG. 16 shows methylation patterns in NDRG4-related CpG islands from targeted bisulfite sequencing data. It shows DNA methylation levels of two CpG islands in NDRG4 (chr16:58,497,033-58,498,595, and chr16:58,535,040-58,535,596). The human genome reference version used was hg19, and data were visualized using the IGV browser. The bar graph indicates the average methylation level of CpG sites in each CpG island.

FIG. 17 shows methylation patterns in SEPT9-related CpG islands from targeted bisulfite sequencing data. It shows DNA methylation levels of three CpG islands in SEPT9 (chr17:75,277,317-75,278,172, chr17:75,368,688-75,370,506, and chr17:75,447,477-75,447,821). The human genome reference version used was hg19, and data were visualized using the IGV browser. The bar graph indicates the average methylation level of CpG sites in each CpG island.

FIG. 18 shows methylation patterns in BMP3-related CpG islands from targeted bisulfite sequencing data.

FIG. 19 shows methylation patterns in IKZF1-related CpG islands from targeted bisulfite sequencing data.

### DETAILED DESCRIPTION

Hereinafter, the present invention will be described in more detail with reference to examples. These examples serve merely to illustrate the present invention in more detail, and it will be apparent to those skilled in the art that the scope of the present invention according to the subject matter of the present invention is not limited by these examples.

### Examples

### Experimental Methods

Analysis of Infinium HumanMethylation450 BeadChip Data from TCGA

To select candidate genomic DNA regions for targeted bisulfite sequencing, Infinium HumanMethylation450 BeadChip data from TCGA were downloaded from the repository of five major gastrointestinal cancers, namely, colon adenocarcinoma (COAD), rectal adenocarcinoma (READ), liver hepatocellular carcinoma (LIHC), stomach adenocarcinoma (STAD), and pancreatic adenocarcinoma (PAAD), via the Genomic Data Commons (GDC) Data Portal (https://portal.gdc.cancer.gov/). The beta value of each CpG site was averaged to represent the methylation value of their matched CpG island in accordance with the human genome reference 19 (hg19). The CpG island methylation values of healthy tissue samples were then averaged, and methylation differences between the tumor samples and the average of the healthy tissue samples were tabulated. Finally, the present inventors shortlisted CpG islands that displayed methylation differences between normal and tumor tissues greater than or equal to 20% in more than 20% of the total patients.

Design of Hybridizing Probe Pool

The probe pool was designed according to the manufacturer's instructions. Basic information regarding the target genome is as follows: Application - SeqCap Epi, Organism -Homo Sapiens, Genomic builds - hg19/GRCh37. This was followed by data input in an appropriate BED (browser extensible data) format into NimbleDesign Software (version 4.3; Roche Diagnostics, Rotkreuz, Switzerland). The total number of target regions was 18,834, and the total length of the regions was 23,533,457 bp (probe design No.: IRN4000028910).

Colorectal Tumor and Adjacent Healthy Specimens

A total of 104 colorectal tumors and their adjacent healthy tissues were obtained from Seoul National University Hospital (SNUH; Seoul, Korea). The use of samples was approved by the Institutional Review Board of Seoul National University Hospital and carried out in accordance with the ethical standards and guidelines of the institution (IRB number: 1608-040-784).

Sample Preparation for Targeted Bisulfite Sequencing

Genomic DNA (1 μg) was used to prepare a single targeted bisulfite sequencing library. All genomic DNA of healthy and tumor samples were sheared using a focused ultrasonicator (M220; Covaris, Massachusetts, USA). The quality, quantity, and fragment size (major peak in 250-300 bp) of sheared genomic DNA were verified using a 2100 Bioanalyzer system (G2939BA; Agilent Technologies, California, USA) prior to library preparation. Sheared genomic DNA was then processed through end repair, A-tailing (Kapa Library Prep Kit for Illumina NGS Platform, 7137974001; Roche Diagnostics), and sequencing adaptor ligation steps (SeqCap Adapter Kit A, 7141530001; Roche Diagnostics).

After clean-up with Agencourt AMPure XP beads (A63880, Beckman Coulter, California, USA), the DNA library was bisulfite-converted using the EZ DNA MethylationLightning Kit (D5031; Zymo Research, California, USA) and amplified via precapture polymerase chain reaction (PCR) using KAPA HiFi HotStart Uracil+ReadyMix (NG SeqCap Epi Accessory Kit, 7145519001; Roche Diagnostics) with Pre-LM-PCR Oligo. The quality of the amplified, bisulfite-converted library samples and their sizes (main peak in 250-300 bp) were verified using a Bio-Analyzer. 1 μg of each amplified, bisulfite-converted library was then combined in sets of SeqCap Epi universal and indexing oligos and bisulfite capture enhancer (SeqCap EZ HE-Oligo Kit A, 6777287001; Roche Diagnostics). Each pool was subsequently lyophilized using a DNA vacuum concentrator (Modulspin 31; Hanil Science Co, Ltd.,

Daejeon, South Korea). The dried components were resuspended in hybridization buffer (SeqCap Epi Hybridization and Wash Kit, 5634253001; Roche Diagnostics) and then hybridized with the probe pool (SeqCap Epi Choice S, 7138938001; Roche Diagnostics) for 72 hours at 47° C. Following incubation, libraries were captured (SeqCap Pure Capture Bead Kit, 6977952001; Roche Diagnostics) in a 47° C. water bath and purified at room temperature. Captured bisulfite-converted libraries were amplified via post-capture PCR and then washed with AMPure XP beads. The quality and size (single peak in 250-300 bp) of the libraries were checked using a bioanalyzer, and samples that passed quality control were sequenced on a HiSeq 2500 instrument (Illumina, California, USA) in paired-end mode.

Preprocessing and Preliminary Screening of Targeted Bisulfite Sequencing Data

Trim Galore (version 0.5.0) was used to remove the adaptor sequences from the targeted bisulfite sequencing data. Based on the human CpG island reference hg19 file, bismark was used to align sequencing reads with Bowtie2. The sort and index commands from SAMtools were used. The number of methylated and unmethylated cytosines at each CpG site was listed using a Bismark methylation extractor, and only those 10x or higher were selected and used for downstream analysis.

Finally, the methylation values of CpG sites included in the same CpG island were calculated by averaging the methylation value based on the hg19 reference file. The following analyses were performed based on the assumption that the averaged value represents each respective CpG island.

Targeted bisulfite sequencing data were screened for targets in which DNA methylation increased or decreased by more than 30% in tumor samples compared with healthy tissue samples in 50% or more of the 90 patients. In addition, hypermethylated CpG islands in tumor samples were further filtered to retrieve regions that showed less than 30% DNA methylation in the healthy tissue samples and 50% or greater DNA methylation in the tumor samples. Conversely, hypomethylated CpG islands, in which the average DNA methylation was less than 30% in the tumor samples and more than 50% in the healthy tissue samples, were selected. Finally, the present inventors selected CpG islands where the mean DNA methylation in healthy tissue samples and tumor samples differed by more than 30%.

Analysis of Targeted Bisulfate Sequencing Data

To analyze the CpG site methylation levels in candidate CpG islands from healthy tissue and tumor samples, beta values of CpG sites in candidate CpG islands were extracted using the tabix program of SAMtools (version 1.9), and only the beta values of cytosines in the same strand of adjacent genes were used in the subsequent analysis to identify the optimal MSP target sites. To filter out the low-quality sequencing data, only sequencing data in which the methylation levels of CpG sites were present in 1/3 or more of the total CpG sites in each CpG island were used. Hierarchical clustering with Canberra distance was applied to the methylation level of each sample. Line graphs were also drawn with the same methylation data using ggplot2 (version 3.3.3) and ggsci (version 2.9) in R software. To display the methylation differences of candidate CpG islands between healthy tissue and tumor samples, hierarchical clustering with Manhattan distance was conducted using p-heatmap. Clustering of CRC patients was performed with the methylation data of the three candidate CpG islands in PDXJ, EN2, and MSXJ. Using IGV, the data regarding the average methylation levels of genes in healthy and tumor tissues were visualized.

Analysis of TCGA Colon Adenocarcinoma RNA Sequencing Data

320 read count files (healthy tissue sample=41, tumor sample=279) which had been quantified with HTSeq, were used to analyze the CRC gene expression pattern of healthy tissue and tumor samples. Each read count was integrated into a matrix format, and the list of differentially expressed genes between healthy tissue and tumors was generated using the DeSeq2 package (version 3.12) in R software. Meanwhile, the TPM value of each gene was derived by using the scaled-estimate value from TCGA RNA-seq V2 data. Meanwhile, as genes that showed a greater than 2-fold change, genes with statistical significance (adjusted p-value <0.05) between normal and tumor samples were selected as final candidates.

Kaplan—Meier Survival Estimation

To investigate patient survival according to the expression level of the candidate genes, the present inventors utilized the UALCAN database (http://ualcan.path.uab.edu/index.html). Genes of interest were tabulated in a specified format, and the appropriate cancer type for analysis was preselected. UALCAN results culminated in the categorization of two groups: (1) high expression of queried genes (upper 25%) and (2) low/medium expression of queried genes (lower 75%). To evaluate whether methylation in the intragenic regions of PDXJ, EN2, and MSX1 has the potential as a prognostic marker in CRC, the survival (version 3.2-7), survminer (version 0.4.8), and ggplot2 packages (version 3.3.3) in R software was used. Progression-free survival in cancer recurrence analysis and overall survival (OS) in the survival analysis of CRC patients were evaluated. The statistical significance of the survival ratio was calculated using the log-rank test.

Overexpression Construct

Each overexpression construct of the candidate genes was subcloned from the pcDNA3-NFlag-NLRP3 vector. To obtain insert fragments, the present inventors designed PCR primers that specifically amplified target sequences on HCT116 and SW480 cDNA with reference to the National Center for Biotechnology Information. As the target genes have numerous CpG sites, the melting temperature (Tm) of the target amplicon naturally increases, hindering the PCR reaction. Thus, the present inventors pre-boiled HCT116 and SW480 cDNA for 10 min before commencing PCR to completely separate the double-stranded structure of the template DNA.

Cell Cultures

The colon cancer cell lines HCT116, LoVo, and SW480 were kindly gifted by Prof Sungsoon Fang of Yonsei University South Korea, a healthy colon fibroblast cell line, CCD-18Co was purchased from the Korean Cell Line Bank (KCLB), HCT116, LoVo, and SW480 cells were maintained in RPMI 1640 medium (11875119; Gibco) supplemented with 10% fetal bovine serum (SH30084.03; Hyclone), and CCD-18Co was grown in DMEM (DMEM/High glucose with L-glutamine, sodium pyruvate with phenol Red, SH30243.01; Hyclone) with 10% FBS. All cell lines were incubated at 37° C. and 5% CO2 in a humidified incubator. For overexpression of candidate genes in vitro, HCT116 cells were seeded in 60-mm culture plates and transfected with either an empty vector or a construct with the candidate genes using Lipofectamine 2000 (11668019; Thermo Fisher Scientific, Massachusetts, USA). The transfection efficiency of each overexpression construct was confirmed by the western blotting of the tags. SW480 cells were transfected with the dCas9-TET1 construct using Lipofectamine3000 (L3000015; Thermo Fisher Scientific) to enhance transfection efficiency.

Transfection efficiencies were verified using GFP as detected by fluorescence microscopy (Cell Imaging System, fl_AMF-4306; EVOS). To detect DNA methylation status and mRNA expression level simultaneously, both genomic DNA and total RNA were extracted from a single sample using AllPrep DNA/RNA mini kit (80204; Qiagen) and subjected to qMSP and qPCR, respectively.

Western Blotting

To confirm the overexpression of candidate genes compared to the empty vector, Western blotting was conducted by immunoblotting FLAG-tags at the N-terminus of each construct using antibodies against a-flag (F7425-.2MG; Sigma-Aldrich) and a-GAPDH (SC-25778; Santa Cruz, Texas, USA).

Cell Proliferation Assay

A total of 1×10⁵ HCT116 cells were transfected with the gene construct for 24 h, followed by seeding in 24-well plates. Cell viability was determined by measuring the absorbance at 450 nm using Cell Counting kit-8 (CK04-11; Dojindo, Kumamoto, Japan) and a microplate reader (Molecular Devices, LLC) at 450 nm at the indicated time points.

Invasion Assay

Invasion assays were performed in 24-well transwell plates (8-nin pore size, 3422; Costar). For invasion assays, 2×10⁵HCT116 cells were transfected with the gene construct for 24 hours, followed by seeding in Matrigel-coated upper chambers. The upper chamber was filled with serum-free RPMI medium, while the lower chamber was filled with RPMI medium supplemented with serum as a chemoattractant. After incubation for 48 hours, the cells that had not invaded through the membrane were removed, and the invaded cells were stained with crystal violet and counted.

MSP Primer Design

To validate DNA hypermethylation in candidate CpG islands in vitro, the present inventors used the following criteria to design MSP primers. First, the Tm difference between the forward and reverse primers was less than 2° C. Tm, which was calculated using Oligo Calc (version 3.27), was set between 55° C. and 60° C. Primer length was designated as 22 bp to 33 bp, with the expected PCR amplicon size set between 100 bp and 160 bp^{[25]}. Additionally, with reference to the DNA methylation status in the targeted bisulfite sequencing data, the present inventors designed MSP primers to include at least 6 CpG sites in the primer binding regions. Finally, regions where 2/3 or more of the CpG sites are methylated by less than 20% in healthy tissues, and are methylated by more than 50% in tumors were selected as primer binding targets. MSP primer sets that bind to methylated (Met) or unmethylated (Unmet) CpG sites were designed manually using the above-described criteria. Additionally, the present inventors also included primers that bind to partially methylated CpG sites (Half-Met).

Quantitative Methylation-Specific PCR (qMSP)

Prior to measuring DNA methylation levels of target genes, 500 ng of genomic DNA extracted from colorectal cell lines or CRC patients was treated with sodium bisulfite (EZ DNA Methylation-Lightning Kits, D5031; Zymo Research). Concentration of bisulfite-converted genomic DNA was quantified using a UV spectrophotometer (Nanodrop 2000; Thermo Fisher Scientific). In the qMSP reaction, KAPA SYBR FAST qPCR Master Mix (2X) (KK4608; Kapa Biosystems) was used to enhance the GC-rich PCR with a PCR cycler (LightCycler 480 II; Roche Diagnostics). The crossing point (Cp) value was calculated by directly adjusting the signal threshold. The DNA methylation level of each CpG island was calculated using the following equation:

(Methylation Level)=2^{(Cp of Unmet) - (Cp of Met)}.

CRISPR/dCas9-TET1 construct

gRNA targeting sites within 100 bp of the MSP primer binding site were selected through Chopchopv2 and further filtered for the least number of off-target sites and best targeting efficiency (Labun et al., 2016). The cloning process was conducted according to the gRNA cloning protocol of Mali P (Mali et al., 2013; Morita et al., 2016). Gibson ligation was performed using a cloning kit (639649; Takara Bio Inc., Shiga, Japan), and the cloned gRNA sequence was confirmed by pyrosequencing.

Quantitative PCR (qPCR)

To check the expression of each candidate gene upon demethylation via the dCas9 system, complementary DNA was synthesized from the total RNA using reverse transcriptase (18090050; Invitrogen).

### Experimental Results

Identification of Differentially Methylated Regions in CRC Tissues by Targeted Bisulfite Sequencing

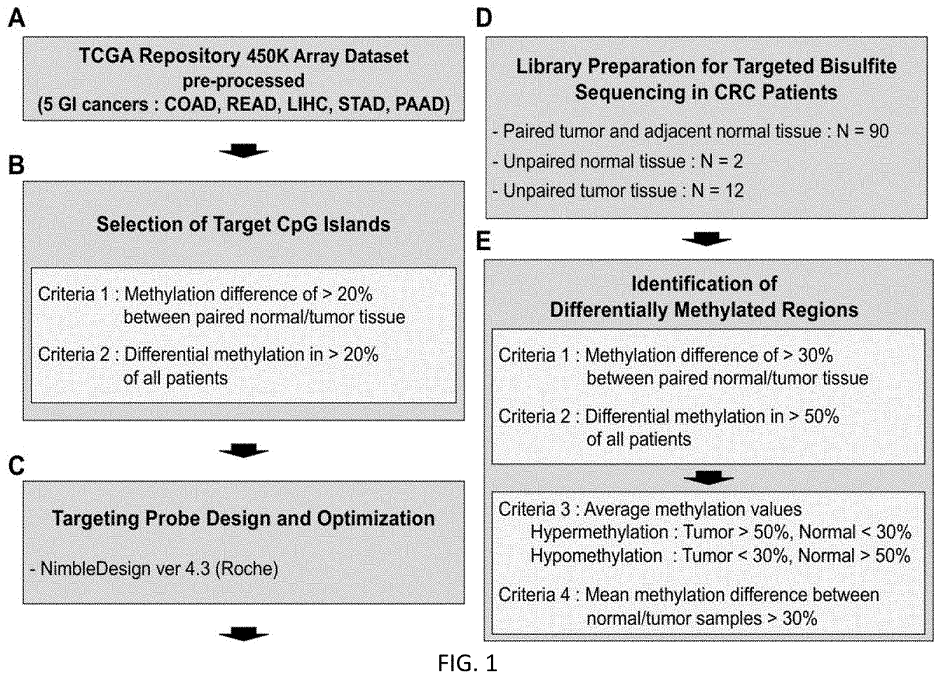

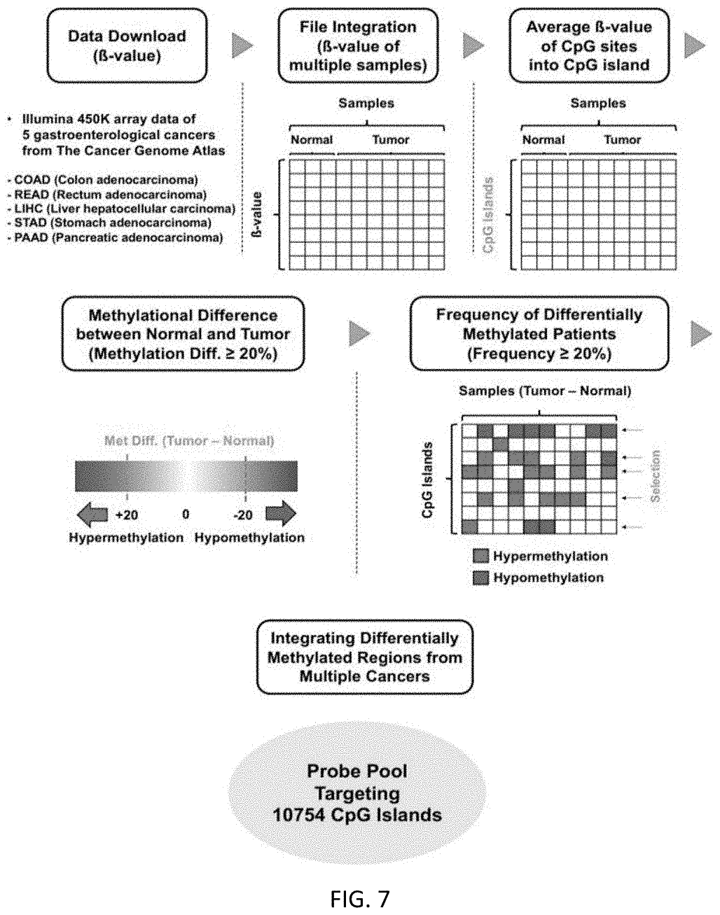

To observe methylation levels in CRC and other types of cancers, 450 K microarray data of five cancer types (COAD, READ, LIHC, AD, and PAAD) were collected from TCGA (FIG. 1A). The beta value of each CpG site was averaged to represent the methylation value of their matched CpG island in accordance with the human genome reference 19 (hg19). The selected CpG islands were further filtered using two criteria. One was that the difference in methylation values between healthy and tumor tissues should be 20% or more, and the other was that such a difference should be present in more than 20% of cancer patients. Therefore, the present inventors obtained 10,754 differentially methylated CpG islands (FIG. 1B and FIG. 7). The selected CpG islands were designed to probe the pool using NimbleDesign (Roche) (FIG. 1C).

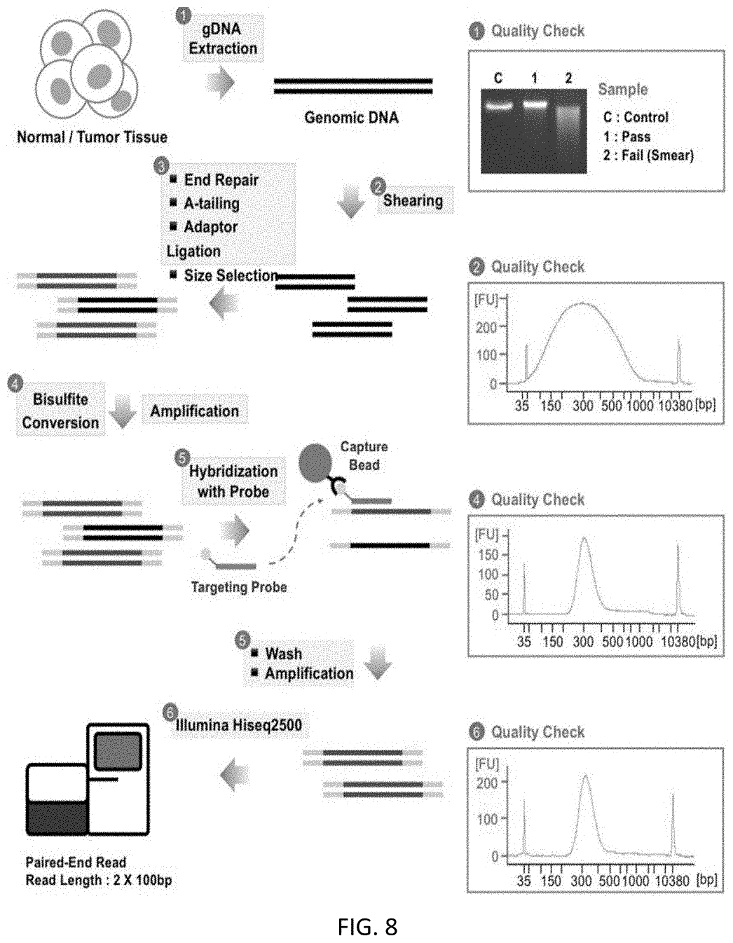

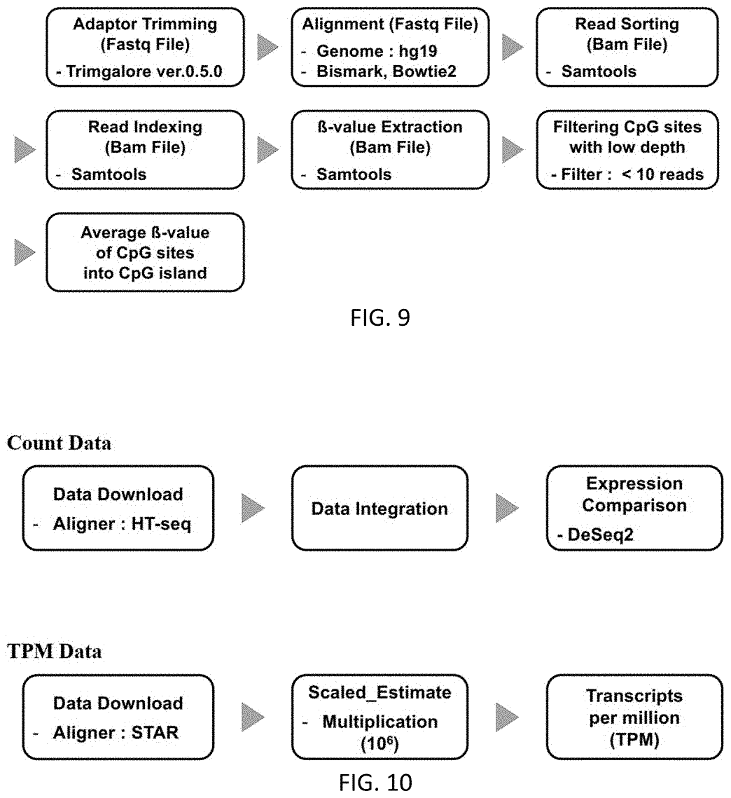

Next, the present inventors performed bisulfite sequencing using the probe pool in CRC tissues. To this end, genomic DNA was obtained from the tissues of 104 Korean CRC patients (90 paired tumors and adjacent healthy tissues, an additional two healthy tissues, and 12 tumor tissues). Targeted bisulfite sequencing libraries were prepared according to the manufacturer's instructions (Roche) (FIG. 1D and FIG. 8), and sequencing was performed. Through targeted bisulfite sequencing of the 194 CRC tissues, the present inventors obtained the beta values of each CpG site, which were averaged to constitute the methylation value of their matched CpG island (FIG. 9). After obtaining the methylation values of CpG islands, more stringent criteria were applied to data. First, the difference in the methylation values of CpG islands between paired healthy and tumor tissues (i.e., from the same patient) had to be more than 30%. Second, this difference had to be present in more than 50% of the patients. Third, even if the difference in methylation values between healthy and tumor tissues was more than 30%, the lower value had to be less than 30%, enabling the easy optimization of MSP by maximizing the signal-to-noise ratio. Finally, to identify the differentially methylated regions that are not specific to some patients, after calculating the overall average of healthy and tumor tissues, the regions with a difference of more than 30% were selected (FIG. 1E).

Thus, the present inventors ultimately identified 40 differentially methylated CpG islands (35 hypermethylated regions+5 hypomethylated regions) in tumor tissues. For instance, the genomic location of chromosome 7:27,147,589-27,148,389 is the intragenic region of HOXA3, where 67 CpG sites are located. On average, the methylation level in this region was 29% in healthy tissues but was 78.7% in tumor tissues. This difference was observed in 83.3% of CRC patients (75 out of 90) (Table 1).

[Table 1] Candidate CpG islands and their matched genes selected from targeted bisulfite sequencing data of 90 CRC patients

| | | | | | | (McaM − |
|---|---|---|---|---|---|---|
| CGI_location | CGI_information | Gene | 30%_Diff | McoM | McaM | McoM) |
| chr7: 27147589-27148389 | Intragenic | HOXA3 | 83.3% (75/90) | 29.0 | 78.7 | 49.7 |
| chr7: 27146069-27146600 | Intragenic | HOXA3 | 82.2% (74/90) | 26.0 | 74.0 | 48.0 |
| chr19: 49669275-49669552 | Intragenic | TRPM4 | 81.1% (73/90) | 24.2 | 73.7 | 49.5 |
| chr2: 54086776-54087266 | promoter | GPR75-ASB3 | 80% (72/90) | 23.9 | 74.3 | 50.3 |
| chr1: 200010625-200010832 | Intragenic | NR5A2 | 78.9% (71/90) | 9.1 | 57.7 | 48.7 |
| chr13: 28498226-28499046 | Intragenic | PDX1 | 72.2% (65/90) | 9.1 | 55.0 | 45.9 |
| chr5: 140857864-140858065 | Intragenic | PCDHGA2 | 72.2% (65/90) | 17.3 | 62.8 | 45.5 |
| chr7: 27182613-27185562 | promoter | HOXA-AS3 | 71.1% (64/90) | 21.4 | 62.6 | 41.2 |
| chr19: 48918115-48918340 | Intragenic | GRIN2D | 69.9% (58/83) | 10.7 | 53.1 | 46.2 |
| chr5: 140864527-140864748 | promoter | PCDHGA2 | 68.9% (62/90) | 9.1 | 52.3 | 43.1 |
| chr5: 134363092-134365146 | Intragenic | PITX1 | 67.8% (61/90) | 21.5 | 59.8 | 38.3 |
| chr7: 158936507-158938492 | promoter | VIPR2 | 65.6% (59/90) | 12.4 | 50.1 | 37.7 |
| chr6: 62995855-62996228 | promoter | KHDRBS2 | 63.3% (57/90) | 11.7 | 51.3 | 39.6 |
| chr6: 10398573-10398812 | Intragenic | TFAP2A | 63.3% (57/90) | 16.1 | 53.0 | 36.9 |
| chr7: 27143181-27143479 | Intergenic | — | 63.3% (57/90) | 26.0 | 62.6 | 36.7 |
| chr7: 24323558-24325080 | promoter | NPY | 63.3% (57/90) | 16.5 | 52.7 | 36.2 |
| chr8: 97171805-97172022 | promoter | GDF6 | 63.3% (57/90) | 19.8 | 53.5 | 33.7 |
| chr13: 53313127-53314045 | promoter | CNMD | 62.2% (56/90) | 15.6 | 50.9 | 35.3 |
| chrX: 142721410-142722958 | promoter | SLITRK4 | 60.7% (54/89) | 19.2 | 54.8 | 35.5 |
| chr7: 155255098-155255311 | Intragenic | EN2 | 60% (54/90) | 17.0 | 52.2 | 35.2 |
| chr13: 102568425-102569495 | promoter | FGF14 | 60% (54/90) | 15.6 | 50.6 | 35.0 |
| chrX: 66766037-66766279 | Intragenic | AR | 58.9% (53/90) | 20.3 | 55.8 | 35.5 |
| chr9: 37002489-37002957 | promoter | PAX5 | 58.9% (53/90) | 22.1 | 56.3 | 34.1 |
| chrX: 101906001-101907017 | promoter | ARMCX5-GPRASP2 | 57.8% (52/90) | 21.6 | 58.2 | 36.6 |
| chr4: 111549879-111550203 | Intragenic | PITX2 | 57.8% (52/90) | 22.9 | 53.7 | 30.8 |
| chr4: 4864456-4864834 | Intragenic | MSX1 | 57.3% (51/89) | 29.7 | 64.3 | 35.3 |
| chr8: 72753874-72754755 | promoter | MSC | 56.7% (51/90) | 26.7 | 58.7 | 32.0 |
| chr19: 46915311-46915802 | Intragenic | CCDC8 | 55.6% (50/90) | 17.7 | 52.1 | 34.5 |
| chr8: 130995921-130996149 | Intragenic | FAM49B | 54.4% (49/90) | 20.9 | 53.1 | 32.1 |
| chr2: 98962873-98964187 | promoter | CNGA3 | 54.4% (49/90) | 19.6 | 51.7 | 32.1 |
| chr2: 5836068-5837643 | Intragenic | SOX11 | 54.4% (49/90) | 20.8 | 51.7 | 30.9 |
| chr11: 65359292-65360328 | Intragenic | EHBP1L1 | 53.3% (48/90) | 26.6 | 58.0 | 31.4 |
| chr6: 108495654-108495986 | Intragenic | NR2E1 | 53.3% (48/90) | 21.5 | 52.0 | 30.5 |
| chr1: 120905971-120906396 | promoter | HIST2H2BA (H2BP1) | 53.3% (48/90) | 28.8 | 59.1 | 30.3 |
| chr13: 70681732-70682219 | promoter | KLHL1 | 50% (45/90) | 25.1 | 55.5 | 30.4 |
| chr16: 87441387-87441671 | Intragenic | ZCCHC14 | 78.9% (71/90) | 77.98 | 28.81 | −49.17 |
| chr7: 5342299-5342599 | Intragenic | SLC29A4 | 77.8% (70/90) | 73.15 | 26.40 | −46.75 |
| chr20: 33762403-33762774 | Intragenic | PROCR | 66.7% (60/90) | 68.94 | 29.90 | −39.04 |
| chr1: 235805318-235805771 | Intragenic | GNG4 | 56.7% (51/90) | 62.69 | 29.03 | −33.66 |
| chr2: 233925091-233925318 | promoter | INPP5D | 57.8% (52/90) | 52.94 | 20.31 | −32.63 |

*McoM: the mean of control (healthy) sample methylation, **McaM: the mean of patient (cancer) methylation.

Selection of Candidate Genes for Developing CRC Biomarkers

The methylation location plays an important role in the correlation between methylation states and gene expression^{[19, 26-28]}. However, while it is well known that hypermethylation in the promoter region inhibits gene expression^{[29]}, the effect of methylation of the intragenic regions on gene expression is still controversial^{[30-36]}

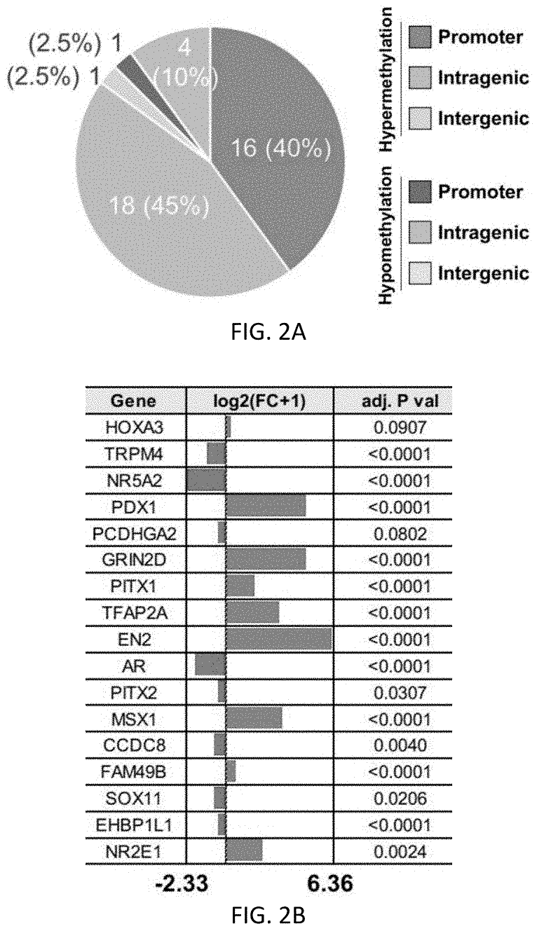

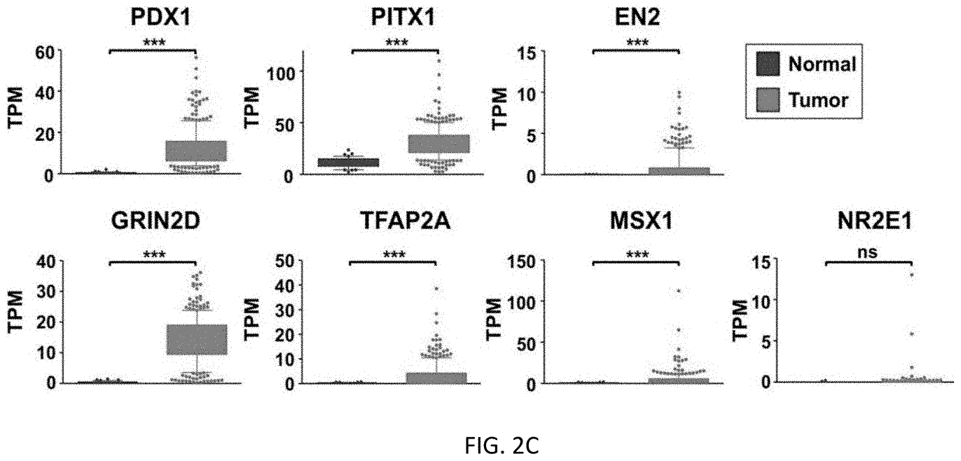

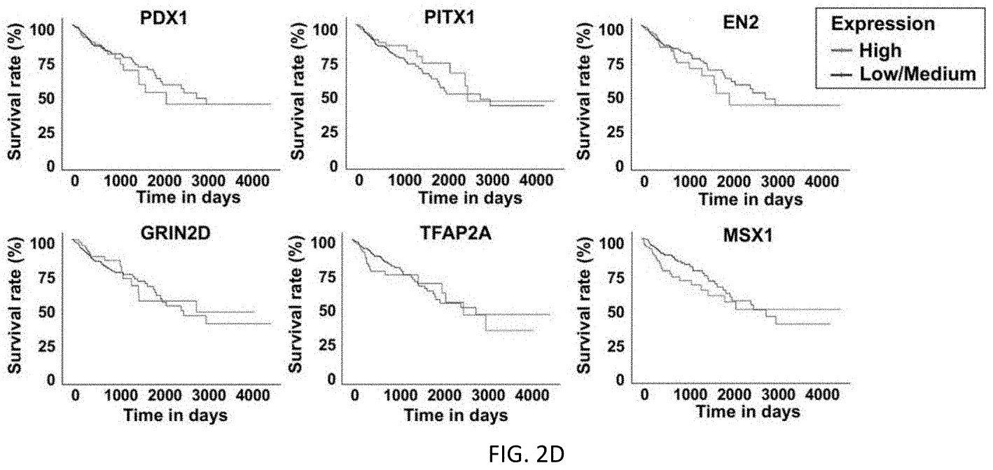

As a result of analyzing the locations of 40 differentially methylated CpG islands, it was observed that, among the 35 hypermethylated regions in the tumor, 16 CpG islands were in the promoter region, 18 were in the intragenic region, and 1 was in the intergenic region. Among the five hypomethylated regions, one was in the promoter region, and four were in the intragenic region (FIG. 2A and Table 1).

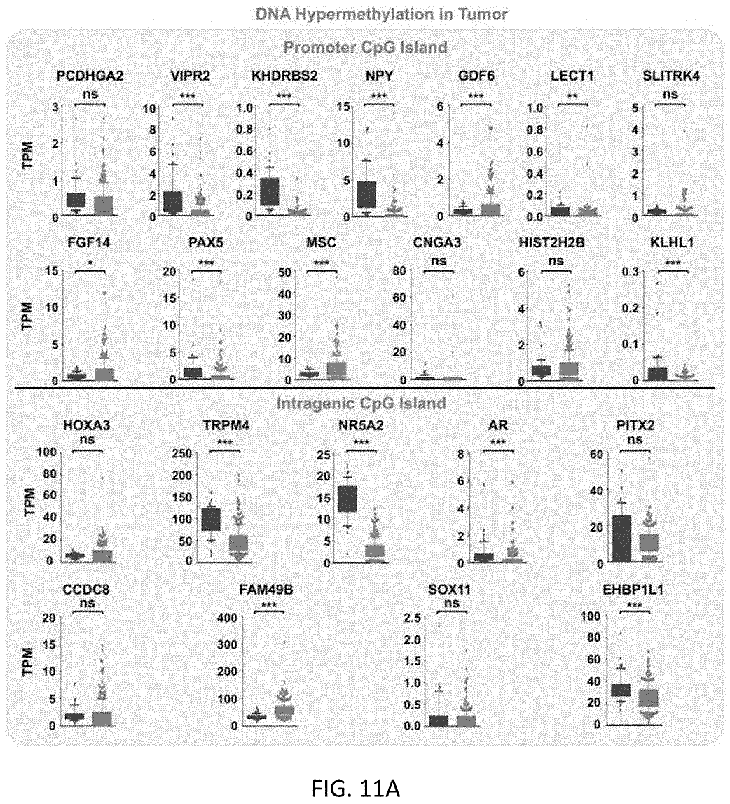

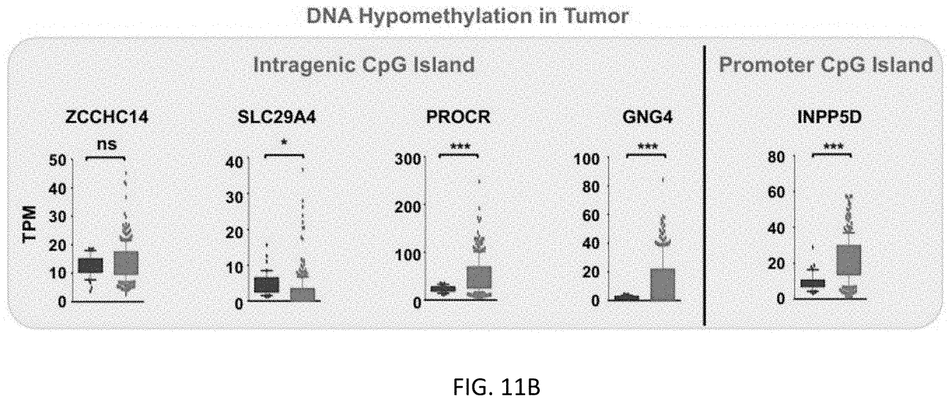

The present inventors next wanted to develop a system to detect methylation states in the 40 differentially methylated CpG islands. To this end, the present inventors examined the regions whose methylation changes have a direct correlation with the expression changes of the related genes. The present inventors speculated that it would be much easier to detect the changes if both methylation and gene expression are increased in tumor tissues compared with healthy tissues. Therefore, the present inventors were interested in the hypermethylated regions, particularly in intragenic regions, because it is difficult to connect the intergenic region to gene expression, and hypermethylation in the promoter is well known to be related to decreased gene expression. To examine gene expression, the present inventors used the TCGA RNA-seq dataset of colon adenocarcinoma (FIG. 10). Among the 18 hypermethylated intragenic regions, two regions were contained in the HOXA3 gene, so the present inventors sought to check the expression of 17 genes. According to the data analyzed by DESeq2, the expression of only seven genes (PDX1, GRIN2D, PITX1, TFAP2A, EN2, MSX1, and NR2E1) was increased by more than two times in tumors (FIG. 2B). To ascertain the level of upregulation of these seven genes, the present inventors also checked the expression of other candidate genes in terms of the TPM value and then excluded NR2E1 due to lack of statistical significance (FIG. 2C and FIGS. 11A and 11B).

Next, the present inventors examined the relationship between the expression of the six genes and the survival rate of CRC patients. According to UALCAN analysis^{[37]}, high expression of PDX1, EN2, and MSX1 was negatively correlated with patient survival (FIG. 2D). Therefore, the present inventors decided to focus on examining these three genes.

Overexpression of PDX1, EN2, or MSX1 Promotes Cell Proliferation and Invasion in Human Colon Cancer Cells

Pancreatic and duodenal homeobox 1 (PDX1) is a critical transcription factor for pancreatic development and beta-cell maturation^{[38]}. PDX1 is overexpressed in pancreatic cancer cells, but its role is different at each cancer stage^{[39-41]}. Although PDX1 has already been reported as a potential cancer marker in CRC, it is based on the observation of PDX1 expression in cancer cells, and its role has not been studied in detail. Homeobox protein engrailed-2 (EN2) is a homeobox-containing transcription factor regulating many developmental stages^{[42]}. Recently, EN2 was reported to play an oncogenic role in tumor progression via CCL20 in CRC^{[43]}. Msh homeobox 1 (MSX1) is also a homeobox-containing transcription factor. MSX1 has been suggested as an mRNA biomarker for CRC, but this suggestion was based on expression pattern observations, and its role has never been demonstrated at the cellular level in CRC^{[44]}.

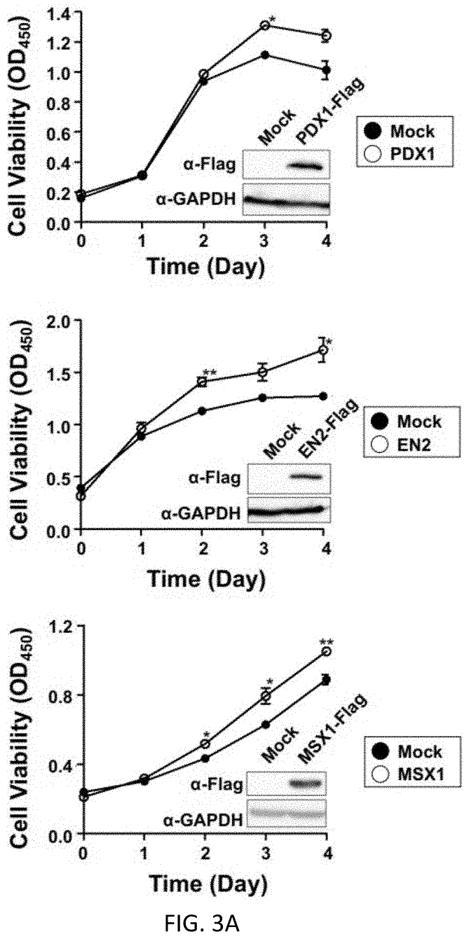

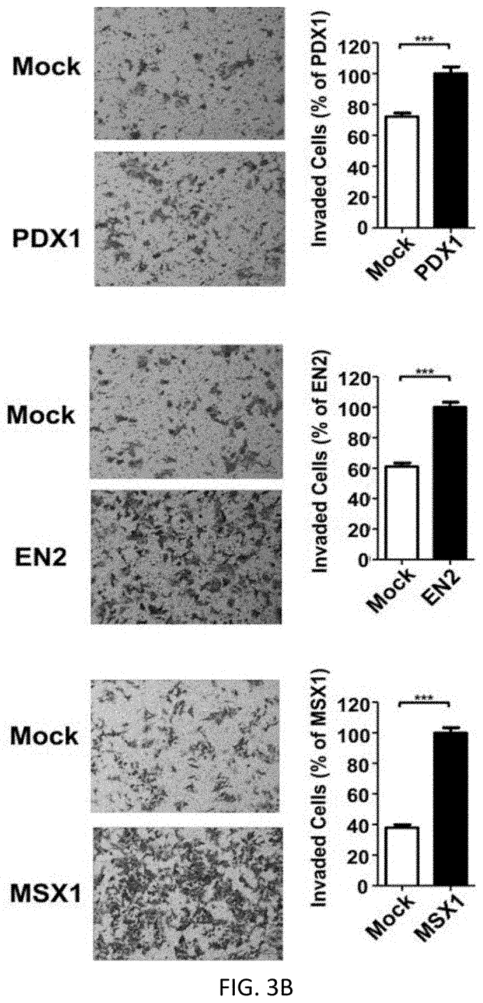

The present inventors transiently transfected each gene into the HCT116 colon cancer cell line and then determined cell proliferation using CCK-8. As a result, overexpression of PDXJ, EN2, and MSX1 increased cell proliferation (FIG. 3A). In addition, as a result of the Transwell assay, it was confirmed that PDXJ, EN2, and MSX1 significantly promoted HCT116 cell migration (FIG. 3B).

Overall, it was concluded that, since the overexpression of PDX1, EN2, and MSX1 is directly related to the proliferation and migration of CRC cells, if the methylation changes in the intragenic regions of these genes are correlated with changes in gene expression, the detection of methylation changes in the marker regions of the present invention would be able to predict cellular conditions.

### Design of MSP Primers for Optimal Detection of Methylation Changes

To detect the methylation changes in the marker regions of the present invention, the present inventors decided to set up a qMSP for each region. Since MSP is a PCR-based experiment, the choice of primer region is very important. If each of the forward and reverse primers has as many CpG sites as possible, the methylation difference between healthy and tumor tissue is large. However, because it would be preferred to perform PCR with methylated primers with unmethylated primers in the same machine, excessive many CpG sites may cause a Tm difference between methylated and unmethylated primers. Finally, the present inventors attempted to make the amplicon length 100 to 160 bp for efficient amplification. Overall, after many trials and errors, the present inventors decided that the forward and reverse primers had at least six CpG sites in total, the Tm of each primer was 55 to 60° C., and the amplicon length was 100 to 160 bp.

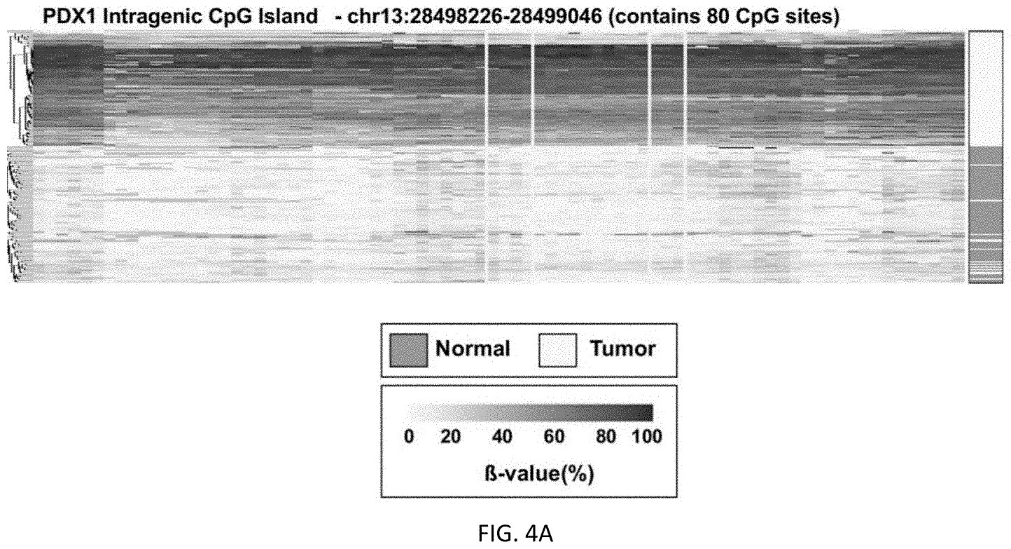

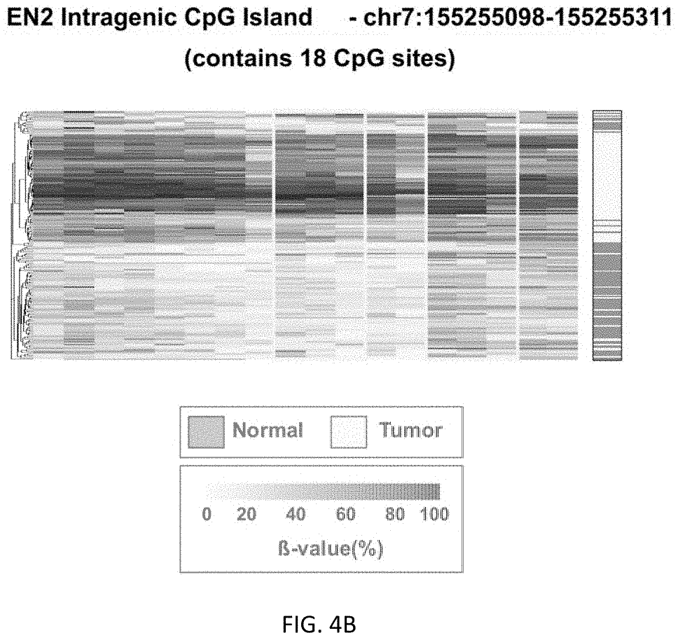

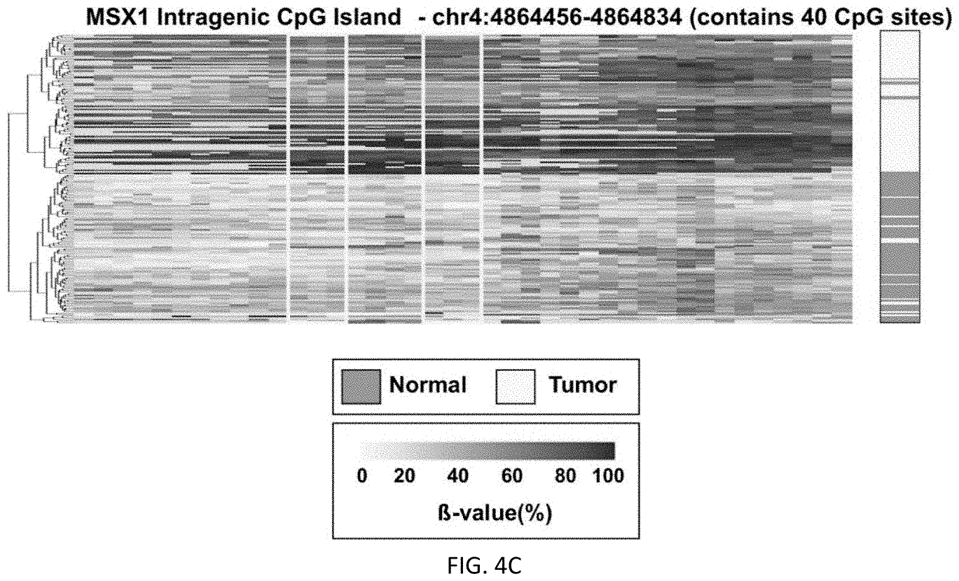

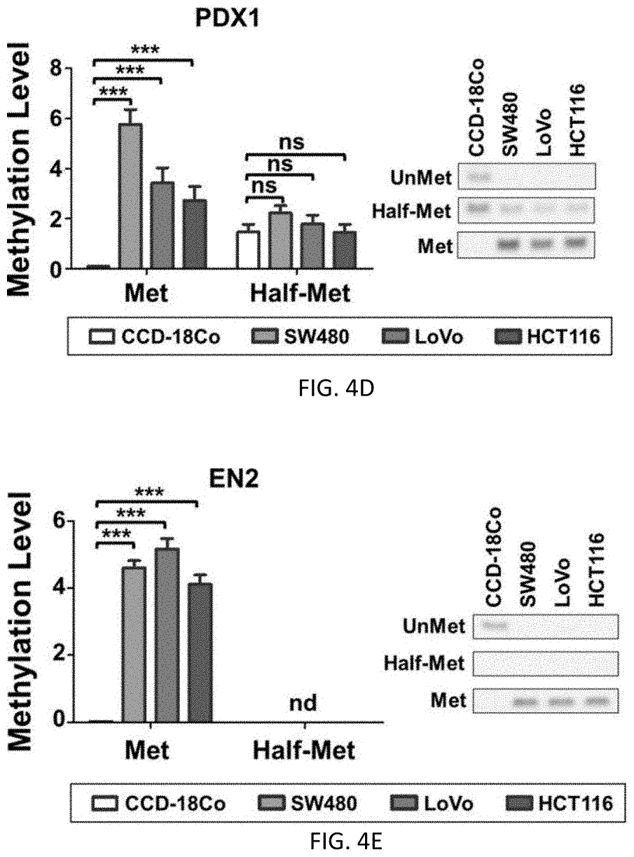

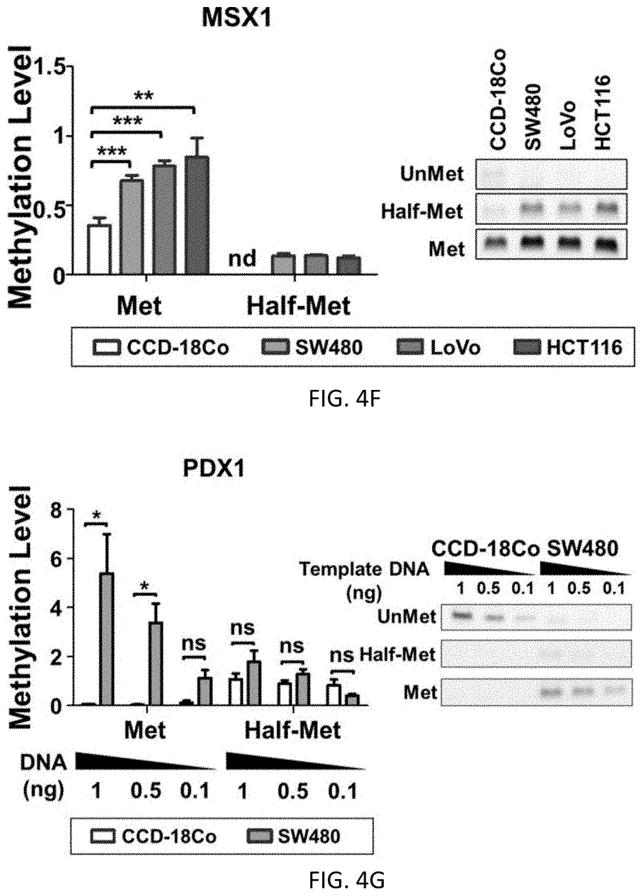

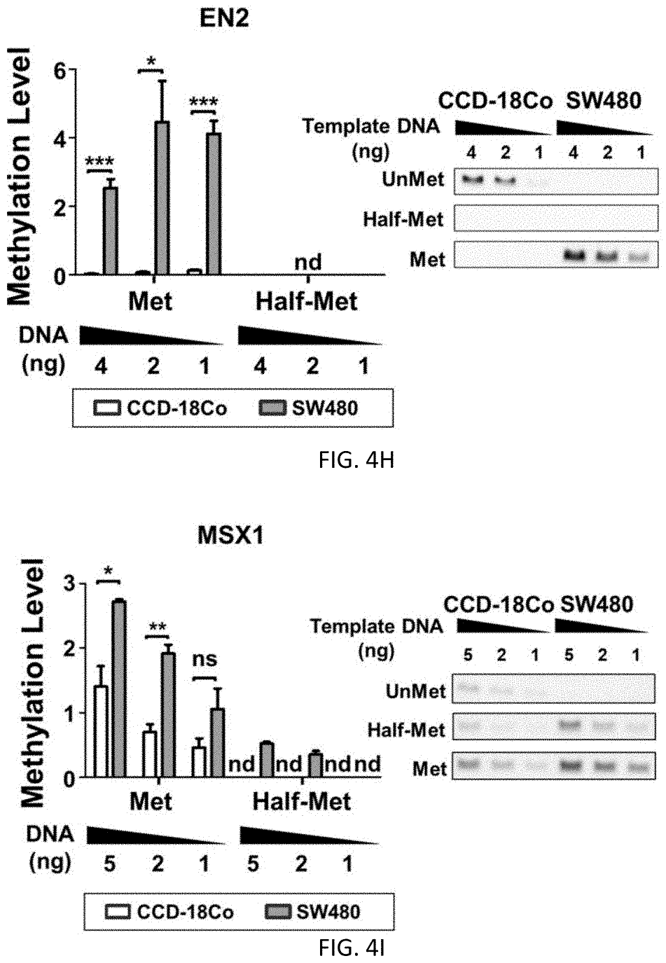

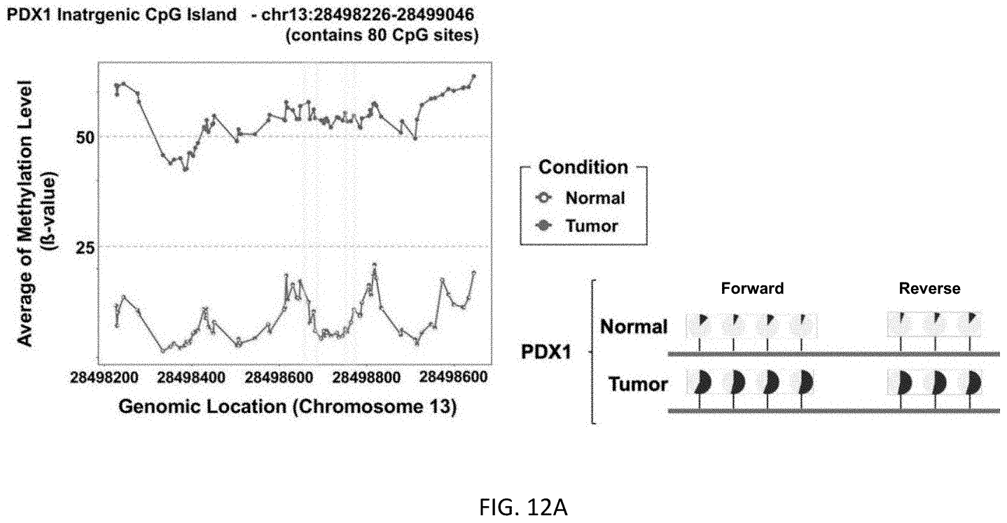

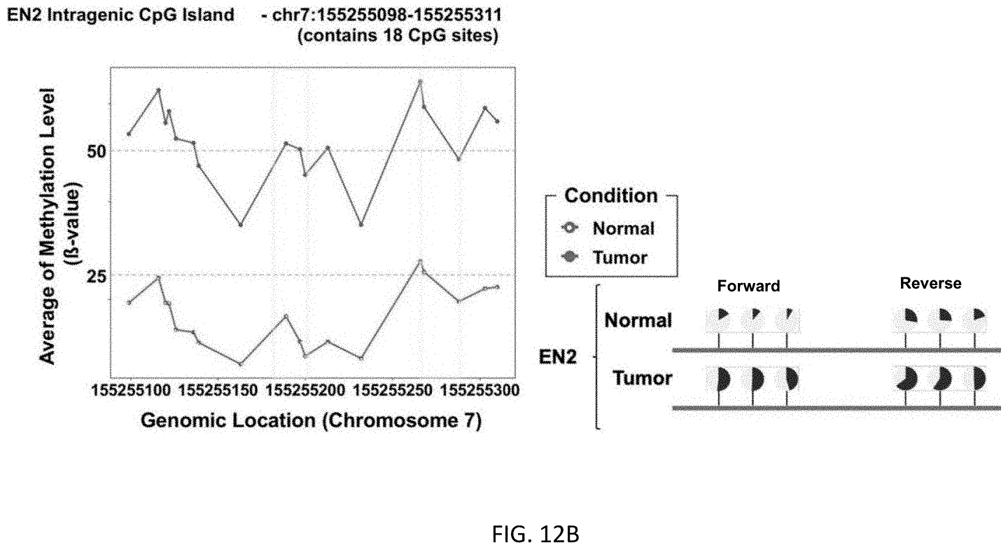

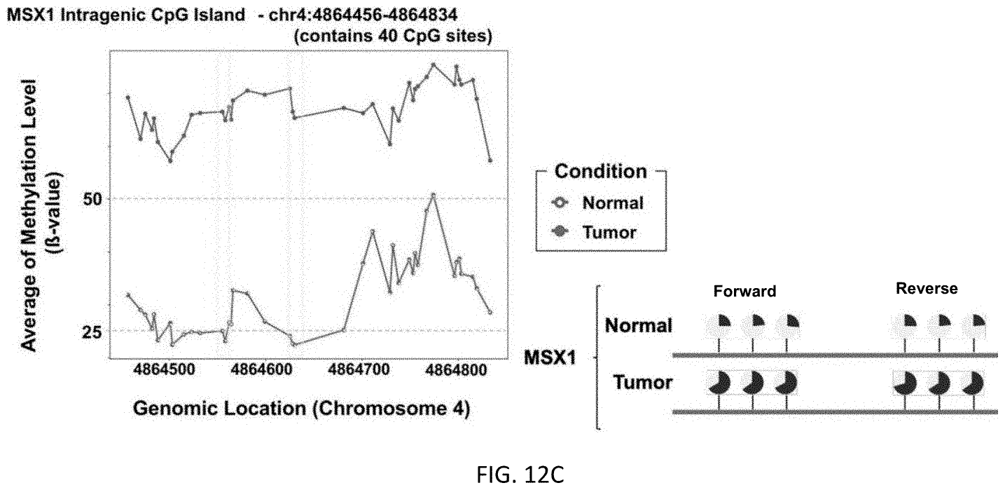

To design MSP primers specific for the intragenic CpG island of PDX1 (chr13:28,498,226-28,499,046), the present inventors examined the methylation changes of 80 individual CpG sites in that region. Although most CpG sites had large differences in methylation changes between tumor and healthy tissues, in an effort to identify the region that satisfies the criteria of the present invention, the present inventors designed MSP primers based on the heatmap and the line graph of the methylation level for each CpG site in the candidate CpG islands (FIGS. 4A and 12A). Since the methylation level of the same strand of the target CpG island is important, the present inventors mainly focused on the methylation level of CpG sites on the sense strand.

The forward primer for PDX1 has four CpG sites, and the reverse primer has three CpG sites. The beta value of these seven CpG sites was approximately 10% in normal tissues but 70% in tumor tissues on average. The amplicon size was 126 bp and 123 bp, and the Tm was 55 to 57° C. (FIGS. 4A and 12A).

For EN2 and MSX1 , MSP primers were also designed in a similar manner. In brief, the forward primer and the reverse primer for EN2 had three CpG sites. The beta value of the six CpG sites was approximately 10% in healthy tissues but 70% in tumor tissues on average.

The amplicon sizes were 127 bp and 112 bp, and the Tm was 57 to 58° C. (FIGS. 4B and 12C). The forward primer and the reverse primer for MSX1 had three CpG sites in each primer. The beta value of the six CpG sites was approximately 10% in healthy tissues but 70% in tumor tissues on average. The amplicon sizes were 151 bp and 144 bp, and the Tm was 55 to 57° C. (FIGS. 4C and 12C).

MSP Primers Efficiently Detect the Methylation States of the Region of Interest

Since the MSP primers of various embodiments of the present invention had a total of six or seven CpG sites, the present inventors not only made a primer set that retained cytosine (methylation primers) or changed all cytosine to thymine (unmethylated primers) but also created a primer set that changed only half of the cytosine to thymine (half-methylation primers). Using these primers, qPCR was performed with bisulfite-treated genomic DNA from the CCD-18Co normal colon cell line and the SW480, LoVo, and HCT116 colon cancer cell lines.

In each CpG island, the methylation primer gave a PCR product in SW480, LoVo, and HCT116 cells but not in CCD-18Co cells. On the contrary, unmethylated primers were detected in CCD18Co cells but not in SW480, LoVo, and HCT116 cells. The half-methylation primer failed to show clear differences among CCD-18Co, SW480, LoVo, and HCT116 cells (FIGS. 4D, 4E and 4F). The methylation level was quantitatively calculated by dividing the methylation primer value or the half-methylation primer value by the unmethylated primer value. SW480, LoVo, and HCT116 cells showed significantly higher methylation levels than CCD-18Co cells when methylation primers were used but not when half-methylation primers were used (FIGS. 4D, 4E and 4F). The present inventors next examined how sensitively the methylation primers could distinguish cancer cells from healthy cells in terms of the amount of template DNA. As a result, the present inventors observed the differential methylation levels of CCD-18Co and SW480 cells via qMSP and found that even 0.5 ng of template DNA, in the case of PDX1, was sufficient to observe the difference (FIGS. 4G, 4H and 41).

From these results, it was confirmed that the MSP primers of the present inventors could distinguish cancer cells from normal cells very efficiently. Although half-methylation primers also have four CpG sites where methylation levels between healthy and cancer cells are different, they could not produce clear differences when MSP was performed, suggesting that only MSP primers have more than enough CpG sites to provide substantially different results.

MSP Primers of the Present Invention could Detect Dynamic Changes in Methylation States

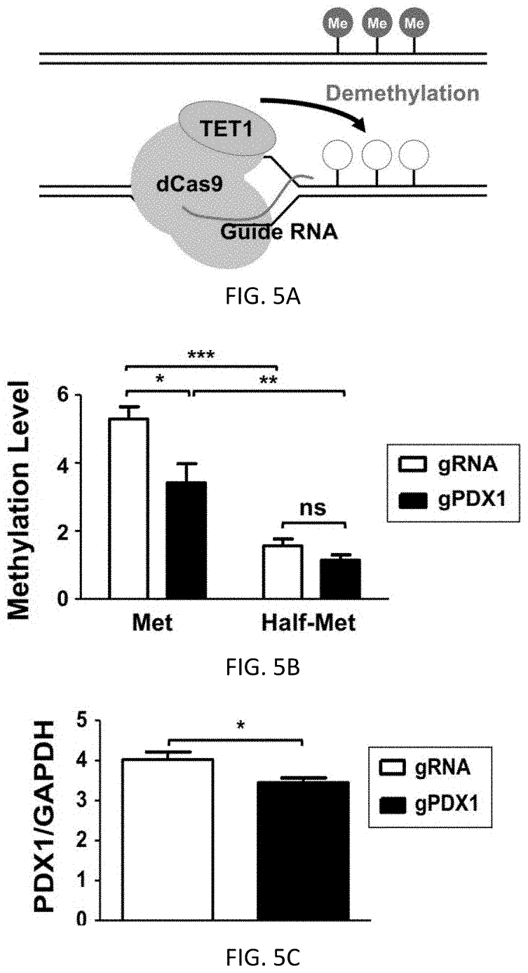

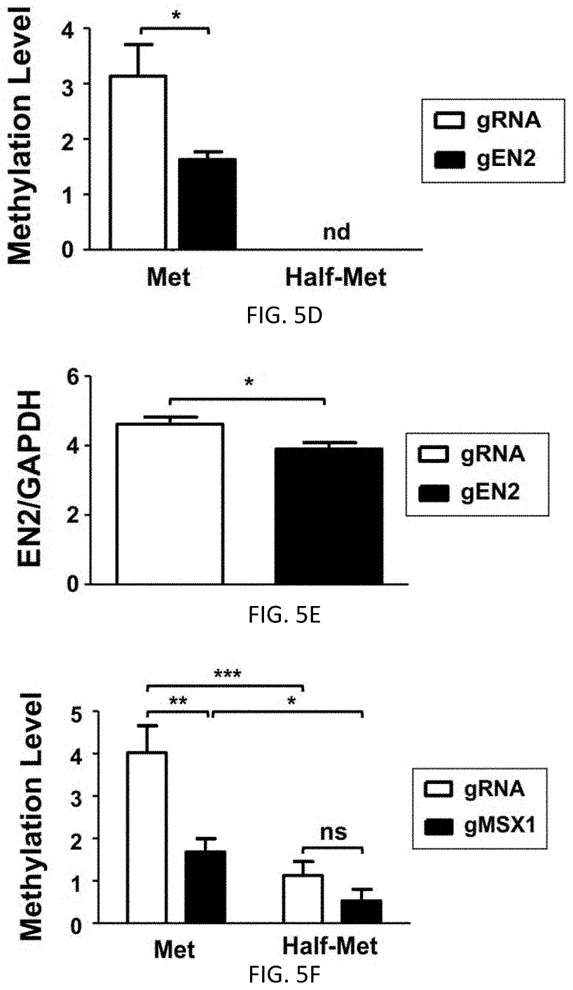

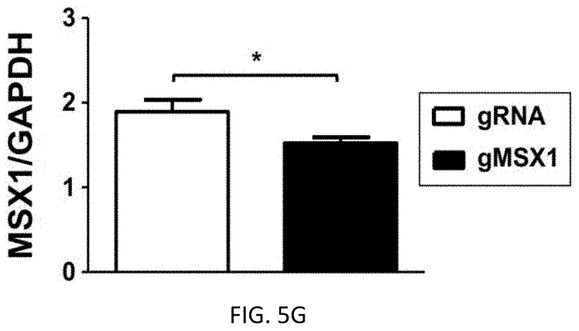

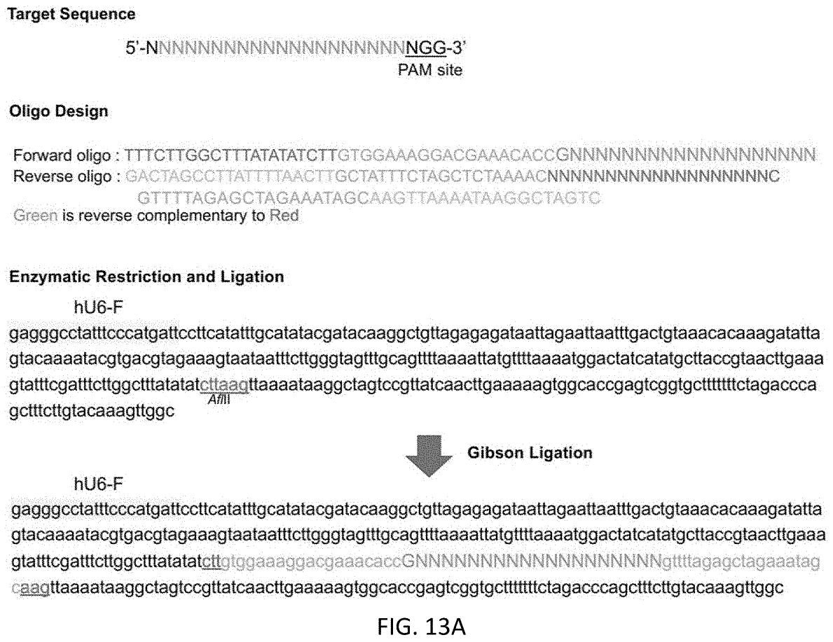

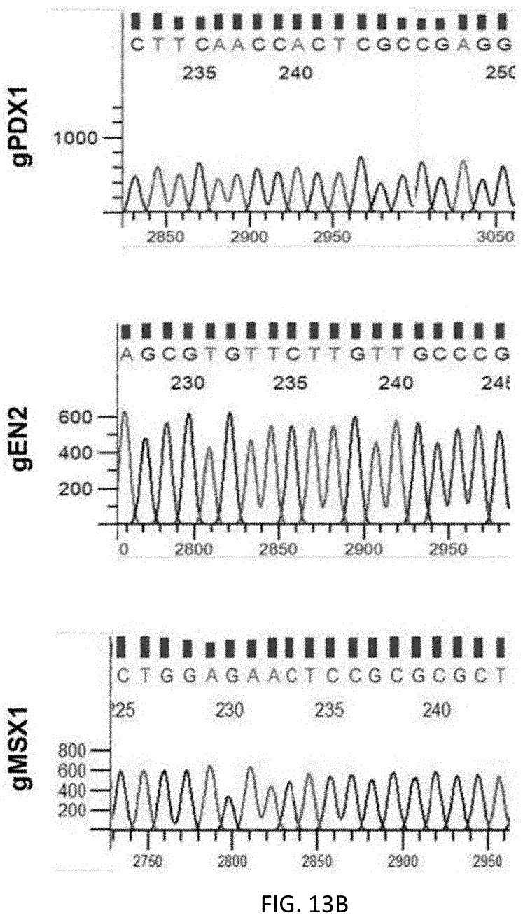

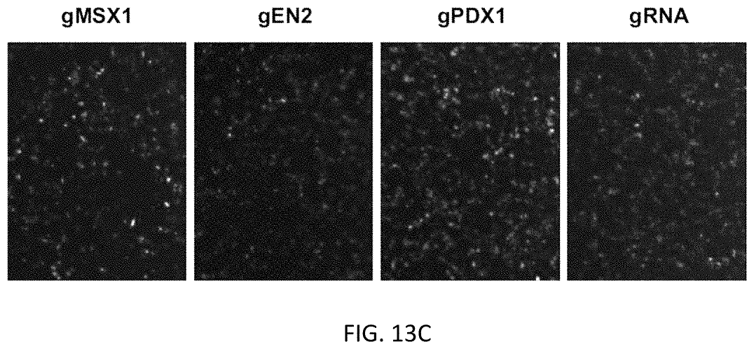

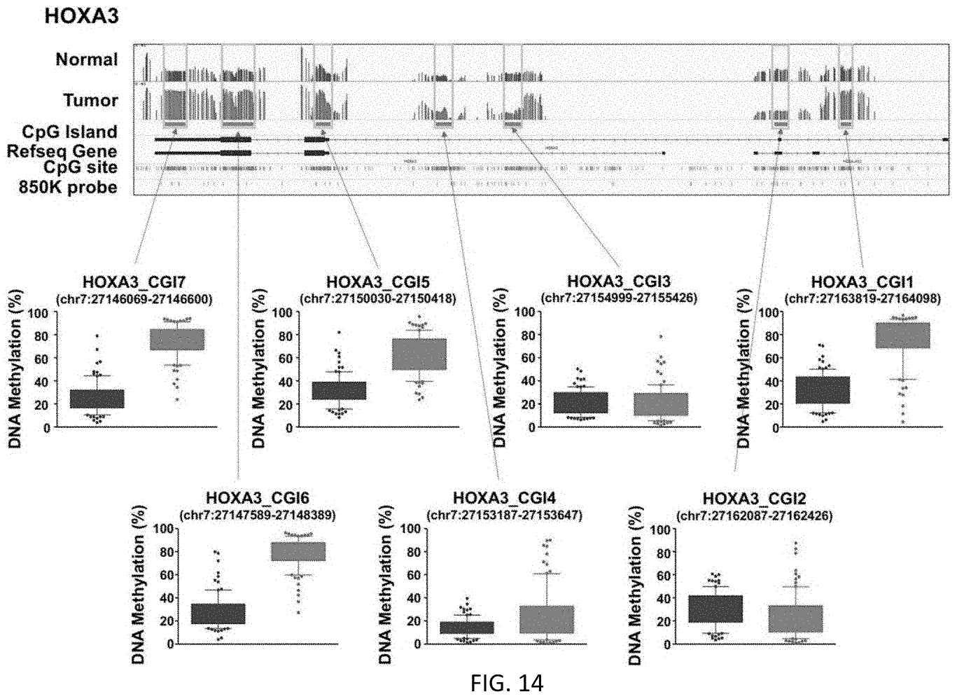

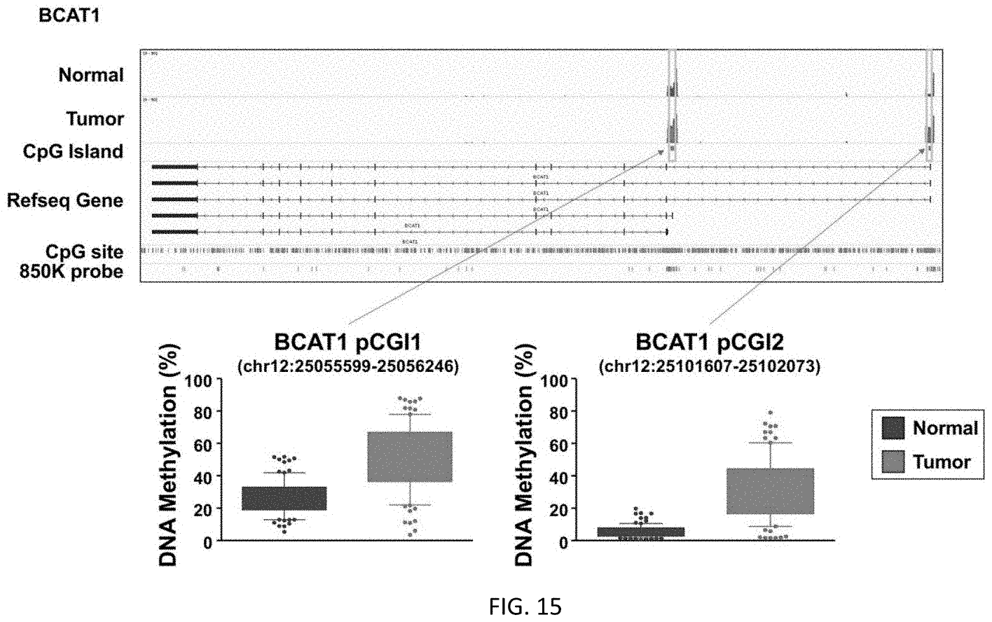

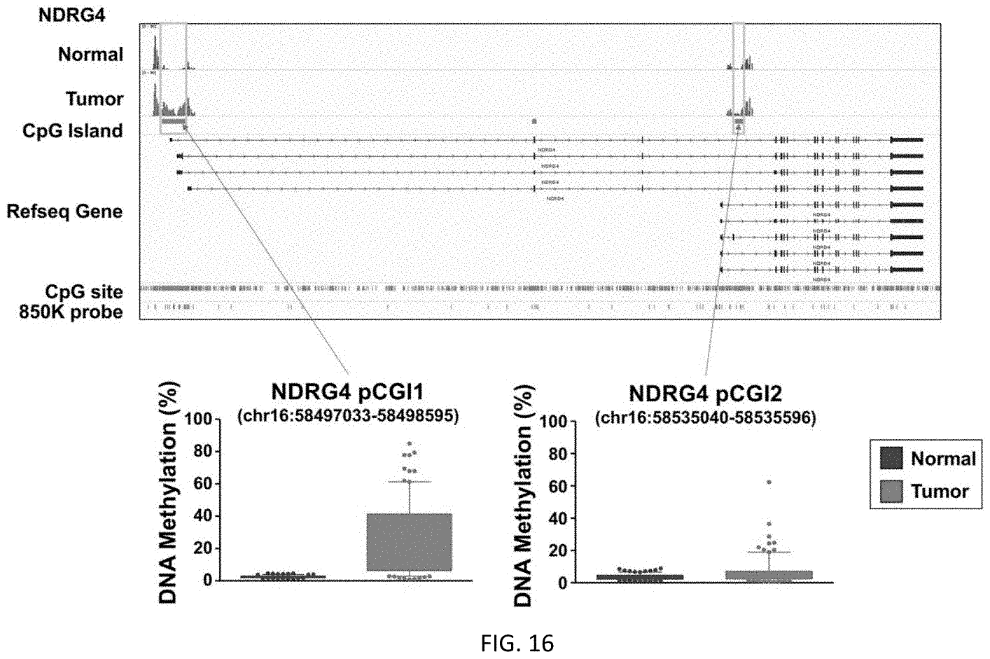

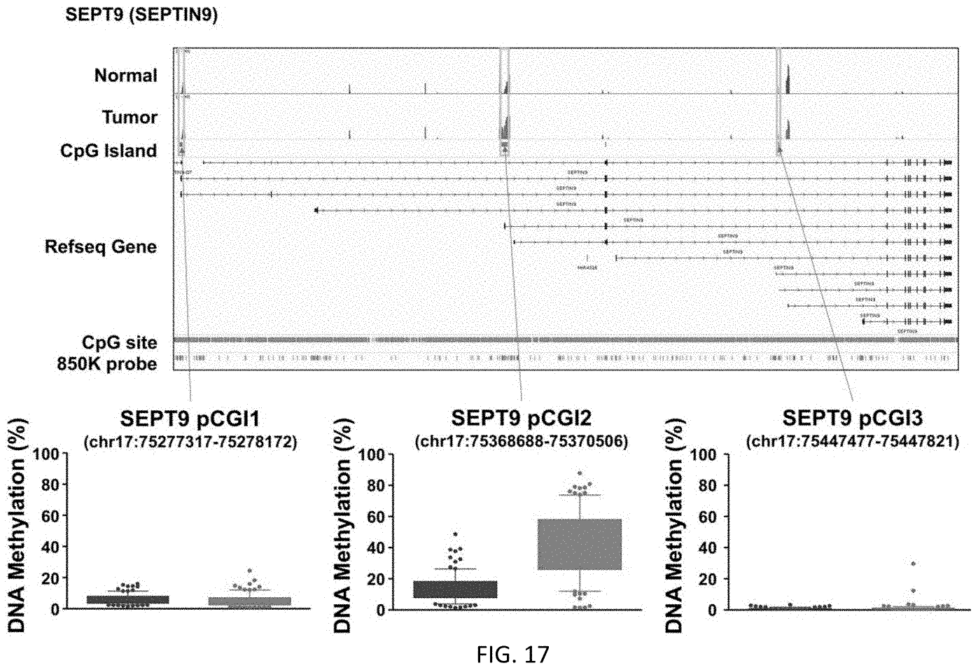

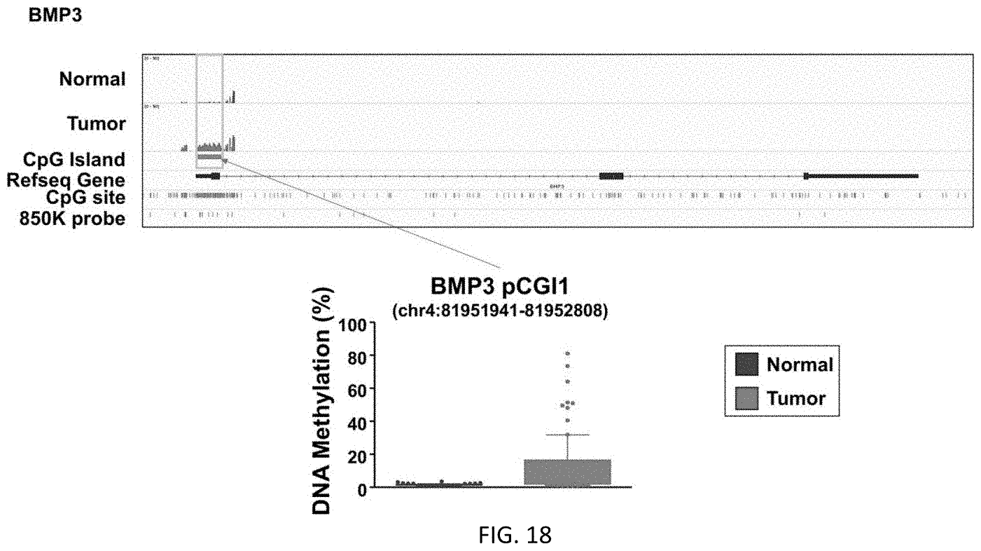

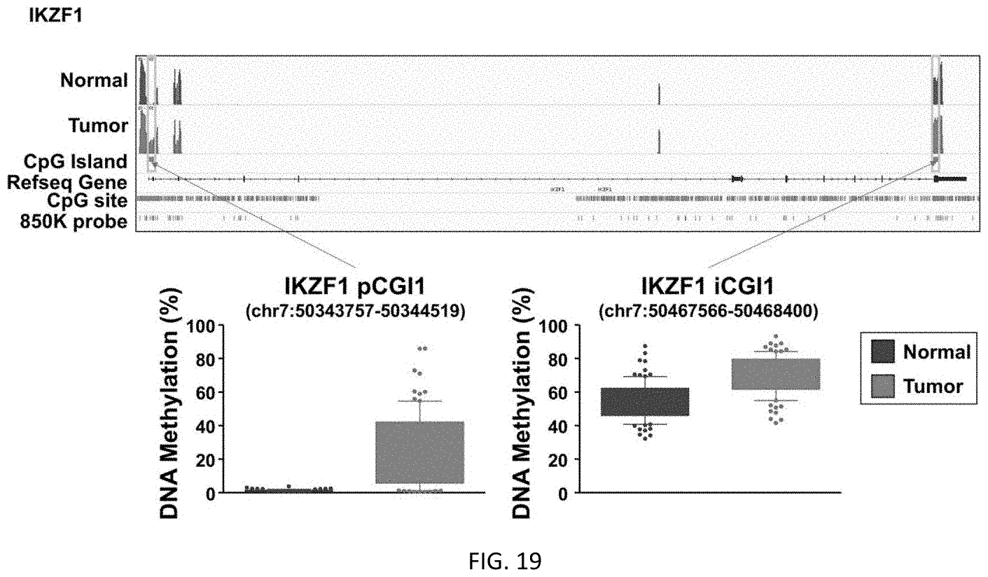

Next, the present inventors examined whether the MSP primers of the present invention could distinguish the dynamic changes in methylation levels out of concern that the data from cell lines might not sufficiently reflect physiological methylation changes due to fixed methylation values. To induce methylation changes, the present inventors used the CRISPR/dCas9-TET1 system (hereafter the dCas9-TET system), which enables to decrease methylation levels in a location-specific manner (FIG. 5A)[^{45]} The gRNA targeting sites within 100 bp of the MSP primer binding site were searched and selected by Chopchopv2 and then the gRNA was subcloned into the dCas9-TET construct (FIGS. 13A and 13B).

After introducing the dCas9-TET system into the PDX1 genomic region (FIG. 13C), the present inventors detected a significant reduction in methylation levels using the methylation primers of the present invention, which contain seven CpG sites. However, the present inventors could not detect this difference using halfmethylation primers (FIG. 5B). The present inventors noted that PDX1 expression was significantly decreased according to the reduction in methylation level in the intragenic region, suggesting that the methylation changes are directly related to gene expression changes (FIG. 5C). The present inventors obtained similar results with EN2 and MSX1 (FIGS. 5D, 5E, 5F and 5G). Thus, it could be confirmed that the methylation primers of the present invention are sensitive enough to detect methylation changes that precede gene expression changes.

Methylation Levels of PDX1, EN2 and MSX1 Predict CRC Metastasis

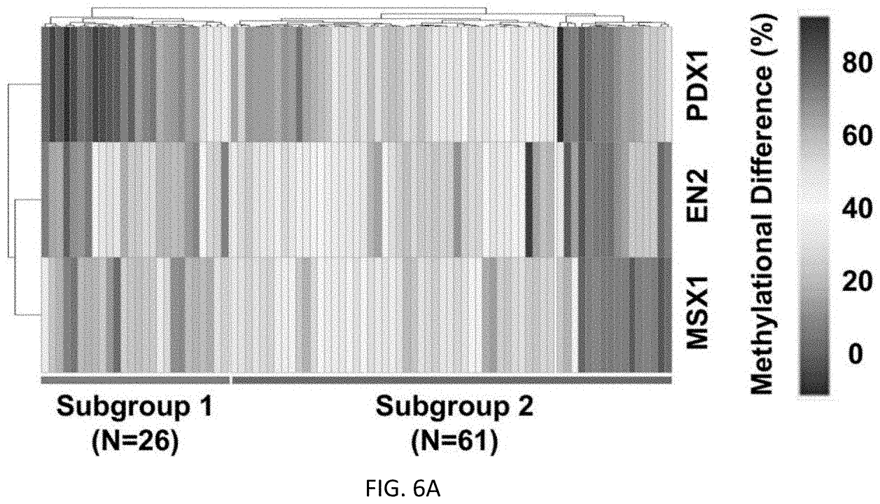

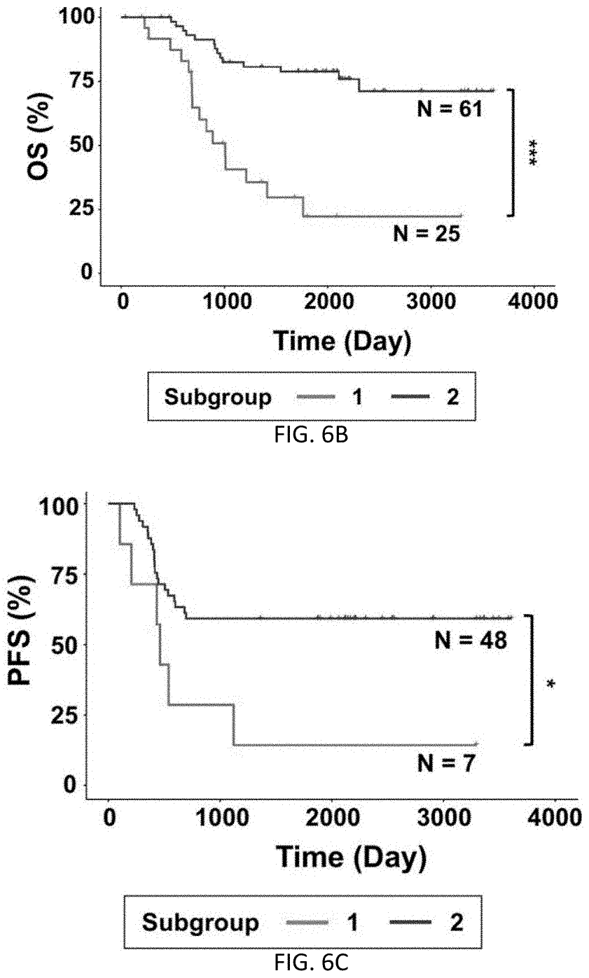

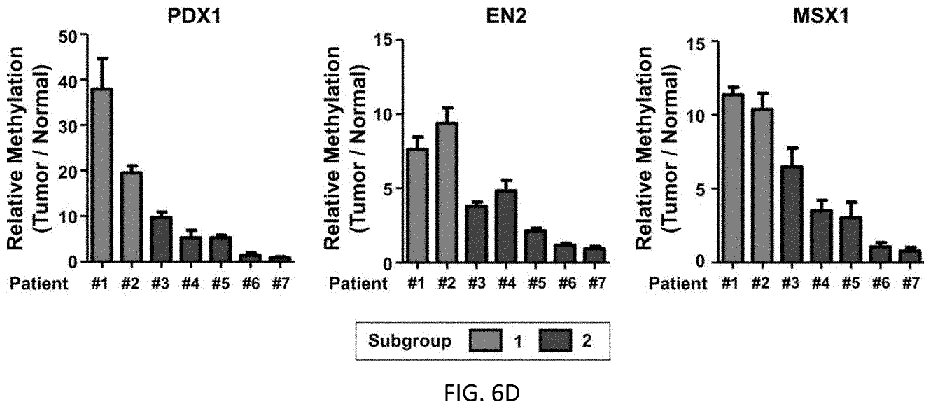

Next, the present inventors examined whether the methylation levels of the intragenic CpG regions of PDX1, EN2 and MSX1 have clinical implications. The present inventors classified patients based on the methylation levels of these regions by conducting hierarchical clustering with the Manhattan distance. Consequently, the present inventors created two groups: the hypermethylated group (Group 1, N=26) and the intermediate methylation and hypomethylated group (Group 2, n=61) (FIG. 6A). Interestingly, these two groups showed a substantial difference in OS (FIG. 6B) and PFS rates (FIG. 6C). In addition, as a result of applying such information to patients, it was confirmed that the majority of stage IV (after metastasis) patients were included in Group 1, whereas the majority of stage III (before metastasis) patients were included in Group 2 (Table 2). Thereby, it was confirmed that PDXJ, EN2 and MSX1 methylation levels could predict CRC patient prognosis.

Finally, the present inventors examined whether the MSP system of the present invention could distinguish between these two patient groups. As a result of performing qMSP using bisulfate-treated genomic DNA from the tumor tissues of seven patients, it was confirmed that two patients in Group 1 showed higher methylation levels in the intragenic regions of PDXJ, EN2 and MSX1 .

[Table 2] Clinical data of the subgroups classified by the methylation level of the intragenic CpG island of PDX1, EN2, and MSX1

| Parameter | Subgroup 1 | | Subgroup 2 | | P |
|---|---|---|---|---|---|
| N | 25 | | 61 | | |
| Age, mean | 58.2 | (40-74) | 63.2 | (36-83) | 0.0343, * |
| (range), year | | | | | |
| Gender | 13:12 | | 39:22 | | 0.304, ns |
| (male:female) | | | | | |
| Stage | n = 25 | | n = 61 | | |
| I | 0% | (0) | 1.64% | (1) | |
| II | 8% | (2) | 0% | (0) | 2.113E−06, *** |
| III | 20% | (5) | 78.7% | (48) | |
| IV | 72% | (18) | 26.2% | (12) | |
| Invasion | n = 25 | | n = 61 | | |
| Lymphatic | 56% | (14) | 45.9% | (19) | 0.0314, * |
| Vascular | 44% | (11) | 19.6% | (8) | 0.00172, ** |
| Perineural | 80% | (20) | 50.8% | (31) | 0.0124, * |
| Differentiation | n = 24 | | n = 58 | | |
| Well | 0% | (0) | 1.7% | (1) | |
| Moderately | 91.7% | (22) | 93.1% | (54) | 0.706, ns |
| Poorly | 8.3% | (2) | 5.2% | (3) | |
| Microsatellite | n = 23 | | n = 58 | | |
| Stable | 91.3% | (21) | 93.1% | (54) | |
| Instable - Low | 4.3% | (1) | 5.2% | (3) | 0.969, ns |
| Instable - High | 4.3% | (1) | 5.2% | (3) | |
| Site of Tumor | n = 25 | | n = 58 | | |
| Ascending | 20% | (5) | 25.9% | (15) | |
| Descending | 4% | (1) | 0% | (0) | |
| Transverse | 4% | (1) | 1.7% | (1) | 0.667, ns |
| Sigmoid | 40% | (10) | 36.2% | (21) | |
| Rectal | 16% | (4) | 20.7% | (12) | |
| Rectosigmoid | 16% | (4) | 15.5% | (9) | |
| Junction | | | | | |

The age of the two subgroups was compared via a two-tailed t test, and the chi-square test was used to analyze the other parameters.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

### REFERENCES

1. Global Cancer Observatory: Cancer Today. [https://gco.iarc.fr/today]
2. Day DW: Scand J Gastroenterol Suppl 1984, 104:99-107.
3. Dekker E, Tanis PJ, Vleugels JLA, Kasi PM, Wallace MB: The Lancet 2019, 394:1467-1480.
4. Vogelstein B, Kinzler KW: Nat Med 2004, 10:789-799.
5. Zecchin D, Boscaro V, Medico E, Barault L, Martini M, Arena S, Cancelliere C, Bartolini A, Crowley EH, Bardelli A, et al: Mol Cancer Ther 2013, 12:2950-2961.
6. Schell MJ, Yang M, Teer JK, Lo FY, Madan A, Coppola D, Monteiro AN, Nebozhyn MV, Yue B, Loboda A, et al: Nat Commun 2016, 7:11743.
7. Xia LC, Van Hummelen P, Kubit M, Lee H, Bell JM, Grimes SM, Wood-Bouwens C, Greer SU, Barker T, Haslem DS, et al: Sci Rep 2020, 10:5009.
8. National Cancer Institute Surveillance E, and End Results Program.: Cancer stat facts: colorectal cancer.
9. Dashwood RH: Oncol Rep 1999, 6:277-281.
10. Force USPST, Bibbins-Domingo K, Grossman DC, Curry SJ, Davidson KW, Epling JW, Jr., Garcia FAR, Gillman MW, Harper DM, Kemper AR, et al: ,L4111,4 2016, 315:2564-2575.
11. Feinberg AP, Vogelstein B: Nature 1983, 301:89-92.
12. Ehrlich M: Oncogene 2002, 21:5400-5413.
13. Rodriguez J, Frigola J, Vendrell E, Risques RA, Fraga MF, Morales C, Moreno V, Esteller M, Capella G, Ribas M, Peinado MA: Canc er Res 2006, 66: 8462-9468.
14. Toyota M, AhujaN, Ohe-Toyota M, Herman JG, Baylin SB, Issa J-PJ: Proceedings of the National Academy of Sciences 1999, 96:8681-8686.
15. Toth, K., Sipos F, K alma A, Patai AV, Wichmann B, Stoehr R, Golcher H, Schellerer V, Tulassay Z, Moln8 B: PLoS One 2012, 7:e46000.
16. A stool DNA test (Cologuard) for colorectal cancer screening. Med Lett Drugs

Ther 2014, 56:100-101.
17. Peterse EFP, Meester RGS, de Jonge L, Omidvari AH, Alarid-Escudero F, Knudsen AB, Zauber AG, Lansdorp-Vogelaar I: J Natl Cancer Inst 2021, 113: 154-161.
18. Koch A, Joosten SC, Feng Z, de Ruijter TC, Draht MX, Melotte V, Smits KM, Veeck J, Herman JG, Van Neste L, et al: Nat Rev Clin Oncol 2018, 15: 459-466.
19. Tse JWT, Jenkins LJ, Chionh F, Mariadason JM: Trends Cancer 2017, 3:698-712.
20. Jain S, Chen S, Chang KC, Lin YJ, Hu CT, Boldbaatar B, Hamilton JP, Lin SY, Chang TT, Chen SH, et al: PLoS One 2012, 7:e35789.
21. Dedeurwaerder S, Defrance M, Calonne E, Denis H, Sotiriou C, Fuks F:

Epigenomics 2011, 3:771-784.
22. Wendt J, Rosenbaum H, Richmond TA, Jeddeloh JA, Burgess DL: Methods Mol Biol 2018, 1708:383-405.
23. Herman JG, Graff JR, MO as, Nelkin BD, Baylin SB: Proc Natl Acad Sci US A 1996, 93:9821-9826.
24. Hernandez HG, Tse MY, Pang SC, Arboleda H, Forero DA: Biotechniques 2013, 55:181-197.
25. Kibbe WA: OligoCalc: Nucleic Acids Res 2007, 35:W43-46.
26. Klutstein M, Nejman D, Greenfield R, Cedar H: Cancer Res 2016, 76: 3446-3450.
27. Lu J, Wilfred P, Korbie D, Trau M: Cancers (Basel) 2020, 12.
28. Ng JM, Yu J: Int J Mol Sci 2015, 16:2472-2496.
29. Suzuki MM, Bird A: Nat Rev Genet 2008, 9:465-476.
30. Maunakea AK, Nagarajan RP, Bilenky M, Ballinger TJ, D'Souza C, Fouse SD, Johnson BE, Hong C, Nielsen C, Zhao Y, et al: Nature 2010, 466:253-257.
31. Lee SM, Lee J, Noh KM, Choi WY, Jeon S, Oh GT, Kim-Ha J, Jin Y, Cho SW,

Kim YJ: Proc Nail Acad Sci USA 2017, 114:E1885-e1894.
32. Krinner S, Heitzer AP, Diermeier SD, Obermeier I, La G, Wagner R: Nucleic Acids Res 2014, 42:3551-3564.
33. Shenker N, Flanagan JM: Br J Cancer 2012, 106:248-253.
34. Kinde B, Wu DY, Greenberg ME, Gabel HW: Proc Natl Acad Sci USA 2016, 113:15114-15119.
35. Arechederra M, Daian F, Yim A, Bazai SK, Richelme S, Dono R, Saurin AJ, Habermann BH, Maina. F: Nat Commun 2018, 9:3164.
36. Greenberg MVC, Bourc′his D: Nat Rev Mol Cell Biol 2019, 20: 590-607.
37. Chandrashekar DS, Bashel B, Balasubramanya SAH, Creighton CJ, Ponce-Rodriguez I, Chakravarthi B, Varambally S: UALCAN: Neoplasia 2017, 19: 649-658.
38. Teo AK, Tsuneyoshi N, Hoon S, Tan EK, Stanton LW, Wright CV, Dunn NR: Stem Cell Reports 2015, 4:578-590.
39. Lin C-P, He L: Annual Review of Cancer Biology 2017, 1:163-184.
40. Boons G, Vandamme T, Ibrahim J, Roeyen G, Driessen A, Peeters D, Lawrence

B, Print C, Peeters M, Van Camp G, Op de Beeck K: Cancers (Basel) 2020, 12.
41. Vinogradova TV, Sverdlov ED: PDX1: Biochemistry (Mosc) 2017, 82:887-893.
42. Brunet I, Weinl C, Piper M, Nature 2005, 438:94-98.
43. Li Y, Liu J, Xiao Q, Tian R, Zhou Z, Gan Y, Li Y, Shu G, Yin G: Cell Death Dis 2020, 11:604.
44. Sun AJ, Gao HB, Liu G, Ge HF, Ke ZP, Li S: J Cell Physiol 2017, 232: 1879-1884.

What is claimed is:

1. A method for diagnosing or predicting prognosis of colorectal cancer, comprising measuring a methylation level in an intragenic region of at least one gene selected from the group consisting of PDX1, EN2 and MSX1 genes.

2. The method of claim 1, wherein measuring the methylation level in the intragenic region of PDX1 is carried out by using a methylation-specific PCR (MSP) primer set that specifically recognizes the intragenic CpG island of PDX1.

3. The method of claim 2, wherein the intragenic CpG island of PDX1 comprises the nucleotide sequence of SEQ ID NO: 1.

4. The method of claim 3, wherein the MSP primer set is a pair of primers comprising the nucleotide sequence of SEQ ID NO: 4 and the nucleotide sequence of SEQ ID NO: 5.

5. The method of claim 1, wherein measuring the methylation level in the intragenic region of EN2 is carried out by using a methylation-specific PCR (MSP) primer set that specifically recognizes the intragenic CpG island of EN2.

6. The method of claim 5, wherein the intragenic CpG island of EN2 comprises the nucleotide sequence of SEQ ID NO: 2.

7. The method of claim 6, wherein the MSP primer is a pair of primers comprising the nucleotide sequence of SEQ ID NO: 6 and the nucleotide sequence of SEQ ID NO: 7.

8. The method of claim 1, wherein measuring the methylation level in the intragenic region of MSX1 is carried out by using a methylation-specific PCR (MSP) primer set that specifically recognizes the intragenic CpG island of MSX1 .

9. The method of claim 8, wherein the intragenic CpG island of MSX1 comprises the nucleotide sequence of SEQ ID NO: 3.

10. The method of claim 9, wherein the MSP primer is a pair of primers comprising the nucleotide sequence of SEQ ID NO: 8 and the nucleotide sequence of SEQ ID NO: 9.

11. A method for treating colorectal cancer in a subject, the method comprising:
diagnosing or predicting prognosis of colorectal cancer in the subject by the method of claim 1; and then based on the diagnosis or prediction of prognosis, treating the subject for colorectal cancer.

12. A method for diagnosing colorectal cancer, comprising measuring an expression level of at least one gene selected from the group consisting ofPDX1, GRIN2D, PITX1, TFAP2A, EN2 and MSX1 genes.

13. A method for treating colorectal cancer in a subject, the method comprising:
diagnosing colorectal cancer in the subject by the method of claim 12; and then based on the diagnosis, treating the subject for colorectal cancer.

14. A method for predicting prognosis of colorectal cancer, comprising measuring an expression level of at least one gene selected from the group consisting of PDXJ, EN2 and MSX1 genes.

15. The method of claim 14, wherein the prognosis comprises metastasis of colorectal cancer.

16. A method for treating colorectal cancer in a subject, the method comprising:
predicting prognosis of colorectal cancer colorectal cancer in the subject by the method of claim 14; and then
based on the predicted prognosis, treating the subject for colorectal cancer.

17. A nucleic acid molecule for detecting methylation in a target gene, comprising forward and reverse primers which form an amplicon having a length of 90 bp to 170 bp, comprise a total of 6 to 9 CpG sites in target gene-binding regions thereof, and have a melting temperature (Tm) of 53 to 62° C.

18. The nucleic acid molecule of claim 17, wherein the forward and reverse primers have a length of 20 bp to 35 bp.

19. The nucleic acid molecule of claim 17, wherein a melting temperature (Tm) difference between the forward and reverse primers is less than 2° C.

20. The nucleic acid molecule of claim 17, wherein the primers are used to measure a methylation level in an intragenic region of the target gene.

21. The nucleic acid molecule of claim 17, wherein the primers are methylation-specific PCR (MSP) primers that specifically recognize an intragenic CpG island.

22. The nucleic acid molecule of claim 21, wherein the methylation level in the intragenic CpG island is different between a patient with a cancer and a normal person.

23. A methylation-specific PCR (MSP) primer set selected from the group consisting of:
(a) a primer set comprising the nucleotide sequence of SEQ ID NO: 4 and the nucleotide sequence of SEQ ID NO: 5;
(b) a primer set comprising the nucleotide sequence of SEQ ID NO: 6 and the nucleotide sequence of SEQ ID NO: 7; and
(c) a primer set comprising the nucleotide sequence of SEQ ID NO: 8 and the nucleotide sequence of SEQ ID NO: 9.
